(19) United States
(12) Patent Application Publication
(10) Pub. No.: US 2016/0089854 A1
(43) Pub. Date: Mar. 31, 2016

(54) INTERFACE CONTROL OF SEMI-CRYSTALLINE BIOPOLYMER FILMS

(71) Applicant: TUFTS UNIVERSITY, Medford, MA (US)

(72) Inventors: Florenzo G. Omenetto, Lexington, MA (US); Mark Brenckle, Northborough, MA (US); Benedetto Marelli, Somerville, MA (US); David L. Kaplan, Concord, MA (US); Hu Tao, Austin, TX (US)

(73) Assignee: Tufts University, Medford, MA (US)

(21) Appl. No.: 14/893,443

(22) PCT Filed: May 23, 2014
(86) PCT No.: PCT/US2014/039447

§ 371 Date: Nov. 23, 2015

Related U.S. Application Data
(60) Provisional application No. 61/827,546, filed on May 24, 2013.

(51) Int. Cl. B32B 5/26; B32B 37/04; A61L 31/16; B29C 65/02; B29C 65/00; A61L 31/04; B32B 3/04; B32B 37/16

(52) U.S. Cl. CPC B32B 5/26; B32B 3/04; B32B 37/04; B32B 37/16; B29C 65/02; B29C 66/45; B29C 66/71; A61L 31/047; A61L 31/16; B32B 2262/08; B32B 2535/00; B32B 2457/00; B32B 2317/10; B32B 2309/02; B32B 2309/04; B29L 2031/753

(57) ABSTRACT

The present invention provides, among other things, compositions including a first silk fibroin layer, and a second silk fibroin layer, wherein at least a portion of the first silk fibroin layer is directly adhered to at least a portion of the second silk fibroin layer to form a silk-silk interface and methods of making the same.

### CROSS-REFERENCE TO RELATED APPLICATIONS

This application is a National Stage Entry of International Application No. PCT/US2014/039447, filed on May 23, 2014, which claims priority to U.S. provisional patent application Ser. No. 61/827,546, filed May 24, 2013, the disclosure of each of which is hereby incorporated by reference in its entirety.

### BACKGROUND

The control of the interfaces between polymers has been essential for the development of a number of technologies, ranging from photovoltaics to drug delivery, due to interfacial effects on function and mechanical properties of the materials. Thin film formats, in particular, have been of interest for their use as substrates for electronic and optical devices, with optimization critical signal propagation and materials stability. The development of polymer-based technological devices has necessitated a surge in associated fabrication strategies. An adequate understanding of the interfacial properties in such systems is required to meet the challenges inherent to these applications, ranging from electronics to biomedicine.

### SUMMARY

The present disclosure provides, among other things, technologies for adhering amorphous silk surfaces to one another. In some embodiments, provided technologies involve contacting an amorphous silk surface (or portion thereof) of a silk article with at least one amorphous silk counter surface (i.e., a second silk surface), and subjecting the amorphous silk surfaces to reflow conditions so that reflow is induced and the contacting surfaces of the silk article(s) is/are altered. In some embodiments, the counter surface is or comprises an amorphous silk surface (or portion thereof) of a silk article; in some such embodiments, the reflow adheres the contacting and counter silk surfaces to one another, in some embodiments with a bond strength of at least 500 kPa.

In some embodiments, provided technologies permit fabrication of multi-layer silk fibroin compositions (e.g., structures) and materials. Provided compositions may be any shape. While provided compositions may be any application-appropriate shape, in some embodiments, a composition may be square, hexagonal, rhomboid, triangular, circular, or curvilinear.

In some embodiments, provided technologies are applied to an amorphous surface of a silk fibroin article; in some embodiments, provided technologies are applied to less than an entire amorphous surface, for example so that adherence occurs on only part of the contacting surface.

In some particular embodiments, provided technologies adhere silk surfaces to one another so that one or more non-adhered areas is/are bounded by adhered areas, i.e., so that a pocket is defined. In some embodiments, such a pocket may contain or be filled with a gas (e.g., air). In some embodiments, a pocket may contain or be filled with a liquid (e.g., an aqueous or organic liquid, or combinations thereof). In some embodiments, a pocket may contain or be filled with one or more active entities. In some particular embodiments, a pocket may contain or be filled with a biologically active entity. In some particular embodiments, a pocket may contain or be filled with an electrically active entity.

Silk fibroin protein from the silkworm Bombyx mori has shown promise as a biomaterial for a variety of technological applications due to its biocompatibility, resorbability and ease of processing into a number of formats. However, the present invention encompasses the recognition that prior device fabrication work with silk films has been limited in certain aspects because of the lack of optimization of the silk/silk interface. The present invention is based, in part, on the surprising discovery that modulation of thermal reflow properties of multilayer silk fibroin film constructs leads to previously unknown and advantageous interfacial properties. In some embodiments, modulation of thermal reflow properties may allow for control over the water content, glass transition, and/or beta sheet crystallinity of silk fibroin film constructs. It is herein described that modulation of thermal reflow properties leads to control over the mechanical properties at the interface of multilayer constructs. Among the advantages provided by the present invention, herein described is new insight into the interfacial properties of similar semi-crystalline biopolymers, which increase the number of fabrication options for the development of devices at the biological-technological nexus.

In one aspect, the present invention provides multilayered compositions including a first silk fibroin layer and a second silk fibroin layer, wherein at least a portion of the first silk fibroin layer is directly adhered to at least a portion of the second silk fibroin layer via a silk-silk interface. In some embodiments, the silk-silk interface has a bond strength of at least 500 kPA.

In another aspect, the present invention provides multilayered compositions including a first silk fibroin layer, and a second silk fibroin layer, wherein the first and second fibroin layers are directly adhered to one another at one or more contact points therebetween, which adhered contact points define a silk-silk interface. In some embodiments, the adhered contacts points have a bond strength of at least 500 kPa.

In still another aspect, the present invention provides multilayered compositions including a first silk fibroin layer, a second silk fibroin layer, and a device, wherein at least a portion of the first silk fibroin layer is directly adhered to at least a portion of the second silk fibroin layer via a silk-silk interface and the device is located, at least in part, between the first silk fibroin layer and the second silk fibroin layer. In some embodiments, the device is located completely between the first and second silk fibroin layers. In some embodiments, the device is encapsulated within a pocket. In some embodiments, the device is selected from a sensor, a transmitter, antenna, transistor, any microelectronic component, optoelectronic components such as LEDs, VCSELs, integrated microlasers, and/or a receiver.

In some embodiments, the silk-silk interface has a bond strength of at least 500 kPa, at least 1,000 kPa, at least 1,500 kPa, at least 2,000 kPa, or at least 2,500 kPa.

In some embodiments, the silk-silk interface defines a boundary around non-adhered portion of the first and second silk fibroin layers, thereby defining a pocket.

In some embodiments, the multilayered composition further comprises a third silk fibroin layer wherein at least a portion of the third silk fibroin layer is directly adhered to at least a portion of at least one of the first silk fibroin layer and second silk fibroin layer via a second silk-silk interface. In some embodiments, the second silk-silk interface defines a boundary around the non-adhered portions of at least one of the first and second silk fibroin layers, thereby defining a second pocket.

In some embodiments, the multilayered composition further comprises a third silk fibroin layer and a fourth silk fibroin layer, wherein at least a portion of the third silk fibroin layer is directly adhered to at least a portion of the fourth silk fibroin layer via an additional silk-silk interface to form an additional pocket. In some embodiments, the first silk fibroin layer and second silk fibroin layer are encapsulated within the additional pocket.

In some embodiments, the pocket (or second or additional pockets) contain or are filled with a gas. In some embodiments, the gas is air. In some embodiments, the pocket (or second or additional pockets) may contain or be filled with more than one gas.

In some embodiments, the pocket (or second or additional pockets) contain or are filled with a liquid. In some embodiments, the liquid is an aqueous liquid or an organic liquid (e.g., an oil). In some embodiments, the pocket (or second or additional pockets) may contain or be filled with more than one liquid.

In some embodiments, the pocket (or second or additional pockets) contain or are filled with an active agent. In some embodiments, an active agent is a biologically active agent. In some embodiments, an active agent is an electrically active agent.

In some embodiments, the multilayered composition further comprises a bioactive compound. In some embodiments, the bioactive compound is located substantially within the pocket (or second or additional pockets). In some embodiments, the bioactive compound is an antibiotic, an antiviral, an antifungal, an anti-thrombotic, and/or a growth factor.

In some embodiments, the multilayered composition comprises a plurality of pockets.

In another aspect, the present invention provides methods for bonding a first silk fibroin layer with a second silk fibroin layer via a silk-silk interface including the steps of contacting a first silk fibroin layer with a second silk fibroin layer, and inducing reflow of silk fibroin of the first silk fibroin layer and silk fibroin of the second silk fibroin layer to generate a silk-silk interface with a bond strength of at least 500 kPa.

In yet another aspect, the present invention provides methods of bonding a first silk fibroin layer with a second silk fibroin layer via a silk-silk interface including the steps of contacting a first silk fibroin layer with a second silk fibroin layer, and inducing reflow of silk fibroin of the first silk fibroin layer and silk fibroin of the second silk fibroin layer so that the first silk fibroin layer and second silk fibroin layer become adhered to one another at one or more contact points between them.

In some embodiments, the silk-silk interface has a bond strength of at least 500 kPa, at least 1,000 kPa, at least 1,500 kPa, at least 2,000 kPa, or at least 2,500 kPa.

In some embodiments, the step of inducing comprises treating with heat, pressure, or combination thereof for a duration of time sufficient to induce reflow of silk fibroin at the silk-silk interface. In some embodiments, the heat is between 75° C. and 150° C.

In some embodiments, the duration of time is between 1 second and 120 seconds. In some embodiments, the duration of time is between 5 seconds and 30 seconds.

In some embodiments, the first silk fibroin layer is not annealed. In some embodiments, the second silk fibroin layer is not annealed.

In some embodiments, the first silk fibroin layer has a first initial crystallinity and first initial water content, and the second silk fibroin layer has a second initial crystallinity and second initial water content wherein the first initial crystallinity and the second initial crystallinity are different; and wherein the first initial water content and the second initial water content are different.

In still another aspect, the present invention provides methods including the steps of providing a multilayered composition comprising a first silk fibroin layer, a second silk fibroin layer, and a device, wherein at least a portion of the first silk fibroin layer is directly adhered to at least a portion of the second silk fibroin layer via a silk-silk interface; placing the multilayered composition in an environment; and activating the device. In some embodiments, the device degrades over a period of time. In some embodiments, the period of time is at least one day, at least one week, or at least one month.

In some embodiments, provided methods further comprise providing a device wherein the device is located at least partially between the first and second silk fibroin layers. In some embodiments, the device is located completely between the first and second silk fibroin layers. In some embodiments, the device is encapsulated within a pocket.

As used in this application, the terms “about” and “approximately” are used as equivalents. Any numerals used in this application with or without about/approximately are meant to cover any normal fluctuations appreciated by one of ordinary skill in the relevant art.

Other features, objects, and advantages of the present invention are apparent in the detailed description that follows. It should be understood, however, that the detailed description, while indicating embodiments of the present invention, is given by way of illustration only, not limitation. Various changes and modifications within the scope of the invention will become apparent to those skilled in the art from the detailed description.

### BRIEF DESCRIPTION OF THE DRAWING

FIG. 1 depicts a schematic of an exemplary silk processing experimental setup, indicating conditions, materials used, and outcomes. Specifically, either one (bottom panels) or two (top panels) silk films are placed in between a polished nickel substrate (w/o a nanoscale topological pattern) and PDMS overlayer for even pressure, and ˜50 Psi is applied to the top while the substrate is heated to 80-120° C. After 5-60 seconds of applied pressure, the films are removed and further analyzed. Colors are used to differentiate the two (identical) films in the schematic.

FIG. 2 depicts a graph of exemplary average heating curves for as-cast silk films processed at different set temperatures over the first 60 seconds of treatment, extracted from 1 f/s thermal video of the cross section of the PDMS/fibroin/Ni stack after ensuring consistent bulk temperature of the fibroin throughout treatment. Error is presented as the shaded region around each curve. The dashed line represents T_{g} of films.

FIG. 3 shows a graph of exemplary thermal gravimetric analysis of treated silk films. Residual water content in laminated silk films with varying treatment temperatures for first 60 seconds of treatment, quantified as the percentage mass lost between 25° C. and 200° C. Data are shown as mean +/− standard deviation.

FIG. 4 shows a graph of exemplary crystallization rates over the first 60 seconds of treatment of cast silk fibroin films for tested temperatures. FTIR scans were quantified for beta-sheet content based on the absorbance of the Amide III band. Mean data are presented. Gray dashed areas represent uncrystallized (<54.5) and crystallized (>61) protein conformations.

FIG. 5 shows an exemplary graph of atomic force microscopy analysis of pre-treated fibroin films imprinted with a nanoscale-patterned grating. Change RMS roughness (Rq) due to the imprinting process, as calculated from topographical data, indicating the degree of replication of the grating with characteristic Rq of ˜57.5 nm, for which the pure Rq is presented. Data are shown for representative pre-treatment induced thermal states, as mean +/− standard deviation. A one-way ANOVA was preformed (p<0.05), and the means were found to be statistically significant.

FIG. 6 shows: panel a) Lap shear bond strength of exemplary silk/silk interfaces with pre-treatment condition. Follows ASTM D3136 with modified geometry, with tensile force applied parallel to the laminated interface. Data are presented as mean +/− standard deviation. A one-way ANOVA was applied, and the means were found to be statistically significant (p<0.05); panel b) Representative SEM (top) and optical (bottom) images of interfacial cross sections of laminated silk films. In the optical images, the top film is doped with melanin, as described in the methods, for contrast. White dashed lines in the SEM micrographs represent the edges of the interfacial region. Scale bars are 20 μm and 200 μm for the SEM and optical micrographs, respectively.

FIG. 7 depicts an exemplary graph of linear fit for atomic force microscopy surface roughness analysis of pre-treated, imprinted films, as a function of reflow time. Reflow time for each condition was estimated as crystallization plateau time minus bound water onset time, based on previously presented data. r² value of the fit was 0.998.

FIG. 8 shows an exemplary graph of linear fit for lap shear bond strength data for pre-treated, laminated films as a function of reflow time. Reflow time for each condition was estimated as crystallization plateau time minus bound water onset time, based on previously presented data. r² value of the fit was 0.936.

FIG. 9 shows an exemplary graph of heat during the silk imprinting process. Black line represents set temperature and white dashed line represents approximate thickness in films used.

FIG. 10 shows an exemplary graph of measured relative crystallinities, independent of temperature, as a function of time. Dashed lines represent threshold for “crystallized” and “uncrystallized” conditions.

FIG. 11 shows a schematic of an exemplary silk fibroin pocket concept and fabrication strategy. Panel (a) shows an exemplary pocket fabrication strategy: three uncrystallized silk films are utilized in pocket fabrication; crystallization of the outer layers renders them water insoluble, while the inner device substrate layer can remain crystallized; sealing the outer edges around the device encapsulates it in a protective pocket of silk fibroin; multi-layer fabrication is carried out by repeating the process with an inner pocket as the device layer. Panel (b) shows an exemplary pocket concept; additional control parameters are possible with the addition of a silk/air/device interface [1] to the silk/device interface of traditional passivation [2].

FIG. 12 shows exemplary mechanical properties of certain embodiments. Panel (a) shows crystallization behavior with increasing heat treatment of silk fibroin films. Crystallinity measured by Amide III quantified ATR-FTIR spectroscopy, and water content measured by TGA. Panel (b) shows ASTM D3136 testing of laminated interfaces with (red) and without (blue) an additional uncrystallized silk film adhesive layer. Panel (c) shows SEM images of interface prior to mechanical testing, showing visible gaps in the samples without adhesive.

FIG. 13 shows exemplary silk/air interface characteristics, and device behavior experiments with multilayer silk membranes. Panel (a) depicts a schematic of multilayer fabrication with controlled interface. Crystallized silk is red, and uncrystallized silk is blue. Panel (b) shows a multilayer membrane cross-section using both an optical and SEM image, as fabricated through utilization of provided lamination methods. Black scale bar represents 1 mm, white scale bars represent 100 μm. Panel (c) shows water penetration through multilayer silk membranes as measured by evaporation from sealed tubes over two weeks. Starred groups were significant to p<0.05 by tukey's test. Means were determined significant by one-way ANOVA. Panel (d) shows sample design for resistor degradation test. Panel (e) shows images of magnesium resistor traces degraded in high relative humidity environment shows uneven degradation by islands. Panel (f) shows resistance of degraded magnesium traces over time with degradation high relative humidity conditions. Experimental results are fit with the existing analytical model for reactive diffusion based magnesium degradation.

FIG. 14 illustrates an exemplary design of a pocket containing a device, and accompanying characteristics including device degradation and enhancement of function. Panel (a) depicts a schematic of sample fabrication for in vitro degradation test. [1] Device consists of 8 mm bilayer metamaterial antenna with magnesium upper layer, crystallized silk substrate, and gold lower layer. [2] Polyimide protection of gold layer prevents device failure due to mechanical disruption of gold. [3] Device encapsulated in silk pockets (0, 1, 2, or 3). [4] Acrylic well placed above pocket and edges are attached with [5] adhesive. [6] Device placed on top of complementary copper transceiver antenna fabricated on [7] PCB base, and attached to network analyzer for constant monitoring of the encapsulated device. During degradation, 1 mL DI water is added to the well. Panel (b) shows a graph of observed device degradation behavior over time, showing loss of resonant response, and slight downfield shift of resonance with swelling of silk substrate. Panel (c) shows a graph of calculated change in quality factor over time for degraded encapsulated device. Each curve is a representative sample from 0, 1, 2, 3 pocket groups. Traces are normalized by dividing by initial value. TSP condition represents 1 layer pocket of equivalent silk thickness to 3 layer condition. Panel (d) shows a graph of the increase in time to rapid degradation with additional pocket protection. Linear fit to R2=0.996. Means are significant by one way ANOVA and Tukey's test p<0.05. Panel (e) shows images of zone of inhibition on bacterial lawns treated with ampicillin loaded silk pockets. Panel (f) shows a graph of the quantification of ZOI for each treatment in panel (e).

FIG. 15 shows an exemplary set up for a silk multilayer interface experiment. Briefly, multilayer compositions as described elsewhere were attached to a plastic tube with adhesive to seal the bottom. After filling with water, a rubber stopper was placed in the opposite end to prevent leaking and evaporation.

FIG. 16 depicts a schematic of an exemplary Mg resistor degradation experiment setup. Briefly, Mg Resistors under test were placed in a sealed acrylic chamber with controlled relative humidity. Control was achieved through use of a feedback controller attached to a humidifier and dessicant pump. Resistance was monitored continually using an ohmmeter.

FIG. 17 shows a schematic and flow diagram of an exemplary fabrication method for test devices used for in situ degradation test. Metal was deposited on both sides of the silk substrate using electron beam evaporation through a stainless steel shadow mask. After removal of the masks, a 15 μm polyimide tape protection layer was applied to the underside of the gold layer to prevent mechanical effects from becoming a confounding factor.

FIG. 18 shows an exemplary graph of linear behavior of water dissipation from silk multilayer interfaces. Volume remaining in each tube was monitored daily over the course of 1 week. Behavior was linear in all cases, regardless of the number of layers in the multilayer silk membrane.

### DEFINITIONS

In order for the present invention to be more readily understood, certain terms are first defined below. Additional definitions for the following terms and other terms are set forth throughout the specification.

Approximately or about: As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Article: As used herein, the term “article” is a manufactured format of a material. In some embodiments, and article may be a block, construct, fabric, fiber, film, foam, gel, implant, mat (e.g., woven and/or non-woven), mesh, needle, particle, powder, scaffold, sheet, or tube. In some embodiments, an article is in a dry (e.g., lyophilized) format. In some embodiments, an article contains a liquid or solvent (e.g., an aqueous or organic liquid); in some such embodiments, the liquid is or comprises water (e.g., as may be present in a gel, such as a hydrogel).

Bioactive: As used herein, the term “bioactive”, or “biologically active” refers to a characteristic of any agent that has activity in a biological system, and particularly in an organism. For instance, an agent that, when administered to an organism, has a biological effect on that organism, is considered to be bioactive. In particular embodiments, where a peptide is bioactive, a portion of that peptide that shares at least one biological activity of the peptide is typically referred to as a “bioactive” portion.

In vitro: As used herein, the term “in vitro” refers to events that occur in an artificial environment, e.g., in a test tube or reaction vessel, in cell culture, etc., rather than within a multi-cellular organism.

In vivo: As used herein, the term “in vivo” refers to events that occur within a multi-cellular organism, such as a human and a non-human animal. In the context of cell-based systems, the term may be used to refer to events that occur within a living cell (as opposed to, for example, in vitro systems).

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

### DETAILED DESCRIPTION OF CERTAIN EMBODIMENTS

As described herein, the present invention provides, among other things, technologies for adhering amorphous silk surfaces to one another. In some embodiments, the present invention provides technologies for inducing or permitting reflow in or on part or all of an amorphous silk surface, in contact with a silk counter surface, so that the surfaces are adhered to one another.

The present invention is based, in part, on the surprising discovery that modulation of reflow properties of silk articles, such as multilayer silk fibroin film constructs, leads to previously unknown and advantageous interfacial properties. In some embodiments, modulation of thermal reflow properties may allow for control over the water content, glass transition, and/or beta sheet crystallinity of silk fibroin film constructs. It is herein described that modulation of thermal reflow properties leads to control over the mechanical properties at the interface of multilayer constructs.

Various aspects of the invention are described in detail in the following sections. The use of sections is not meant to limit the invention. Each section can apply to any aspect of the invention. As used herein and in the claims, the singular forms include the plural reference and vice versa unless the context clearly indicates otherwise. Other than in the operating examples, or where otherwise indicated, all numbers expressing quantities of ingredients or reaction conditions used herein should be understood as modified in all instances by the term “about.” In this application, the use of “or” means “and/or” unless stated otherwise.

At many points below, aspects of the invention are exemplified through discussion and/or use of silk fibroin from the silkworm, Bombyx mori. Those skilled in the art will readily appreciate that, in many cases, teachings provided with respect to such silk fibroin from Bombyx mori are applicable to other forms or types of silk fibroin such as, for example, spider silk such as that from Nephila clavipes and/or genetically engineered silk, such as from bacteria, yeast, mammalian cells, transgenic animals or transgenic plants.

In one aspect, the present invention provides multilayered compositions including a first silk fibroin layer and a second silk fibroin layer, wherein at least a portion of the first silk fibroin layer is directly adhered to at least a portion of the second silk fibroin layer via a silk-silk interface. In some embodiments, the silk-silk interface has a bond strength of at least 500 kPA.

In another aspect, the present invention provides multilayered compositions including a first silk fibroin layer, and a second silk fibroin layer, wherein the first and second fibroin layers are directly adhered to one another at one or more contact points therebetween, which adhered contact points define a silk-silk interface. In some embodiments, the adhered contacts points have a bond strength of at least 500 kPa.

Silk Fibroin Layers/Films

Silk is a natural protein fiber produced in a specialized gland of certain organisms. Silk production in organisms is especially common in the Hymenoptera (bees, wasps, and ants), and is sometimes used in nest construction. Other types of arthropod also produce silk, most notably various arachnids such as spiders (e.g., spider silk). Silk fibers generated by insects and spiders represent the strongest natural fibers known and rival even synthetic high performance fibers. Silk is naturally produced by various species, including, without limitation: Antheraea mylitta; Antheraea pernyi; Antheraea yamamai; Galleria mellonella; Bombyx mori; Bombyx mandarins; Galleria mellonella; Nephila clavipes; Nephila senegalensis; Gasteracantha mammosa; Argiope aurantia; Araneus diadematus; Latrodectus geometricus; Araneus bicentenarius; Tetragnatha versicolor; Araneus ventricosus; Dolomedes tenebrosus; Euagrus chisoseus; Plectreurys tristis; Argiope trifasciata; and Nephila madagascariensis.

Silk fibroin proteins offer desirable material characteristics for a number of applications that take advantage of the nature of biological materials, such as biocompatibility. Silk fibroin of the Bombyx mori silkworm has come of considerable interest in this context, owing to its attractive mechanical (B. D. Lawrence, et al., Journal of Materials Science 2008, 43, 6967-6985; S. Sofia et al., Journal of Biomedical Materials Research 2001, 54, 139-48; L. Meinel et al., Bone 2006, 39, 922-31; H.-J. Jin et al., Biomacromolecules 2002, 3, 1233-9), biological (M. Santin et al., Journal of Biomedical Materials Research 1999, 46, 382-9; E. M. Pritchard et al., Journal of Controlled Release: Official Journal of the Controlled Release Society 2010, 144, 159-67), and optical properties (H. Perry et al., Advanced Materials 2008, 20, 3070-3072; B. D. Lawrence et al., Biomacromolecules 2008, 9, 1214-20) for use in biomedical, optical, electro-optical, industrial and other applications.

According to various embodiments, silk articles, such as silk fibroin layers, may comprise any of a variety of silk fibroin proteins including, but not limited to, those described herein and in WO 97/08315 and U.S. Pat. No. 5,245,012. According to various embodiments, a silk fibroin layer may be made using one or more silk protein solutions. Unless otherwise clearly stated, the terms “silk fibroin layer” and “silk film” are used interchangeably herein.

Silk protein solutions can be prepared by any conventional methods known to one skilled in the art. A brief exemplary process for preparing a silk protein solution is provided in order to provide a better understanding of some of the principles of the present invention. In some embodiments, B. mori cocoons are boiled for about 30 minutes in an aqueous solution (e.g. 0.02 M Na₂CO₃). The cocoons are then rinsed, for example, with water to extract the sericin proteins and the extracted silk is dissolved in an aqueous salt solution. Salts useful for this purpose include, lithium bromide, lithium thiocyanate, calcium nitrate or other chemical capable of solubilizing silk. In some embodiments, a strong acid such as formic or hydrochloric may also be used. In some embodiments, the extracted silk is dissolved in about 9-12 M LiBr solution. Regardless of the specific extraction method(s) used, the salt is consequently removed using, for example, dialysis.

In some embodiments, a silk protein solution may be substantially free of sericin. As used herein, “substantially free of sericin” means that sericin is absent from such a preparation, or present in such a trace amount that it does not affect the subsequent step or steps of silk fibroin processing or its downstream application. In some embodiments, a trace amount of sericin that may be present in a silk fibroin preparation is present in concentrations less than about 0.5%, less than about 0.4%, less than about 0.3%, less than about 0.2%, less than about 0.1%, less than about 0.05%, less than about 0.04%, less than about 0.03%, less than about 0.02%, less than about 0.01%, or lower. In some embodiments, a trace amount of sericin that may be present in a silk fibroin preparation is present in a concentration that is below a detectable threshold by conventional assays used in the art.

In some embodiments, one or more biocompatible polymers are added to a silk protein solution in order to form a silk article (e.g., a silk fibroin layer). Suitable biocompatible polymers compatible with various embodiments of the present invention include, but are not limited to, polyethylene oxide (PEO) (U.S. Pat. No. 6,302,848), polyethylene glycol (PEG) (U.S. Pat. No. 6,395,734), collagen (U.S. Pat. No. 6,127,143), fibronectin (U.S. Pat. No. 5,263,992), keratin (U.S. Pat. No. 6,379,690), polyaspartic acid (U.S. Pat. No. 5,015,476), polylysine (U.S. Pat. No. 4,806,355), alginate (U.S. Pat. No. 6,372,244), chitosan (U.S. Pat. No. 6,310,188), chitin (U.S. Pat. No. 5,093,489), hyaluronic acid (U.S. Pat. No. 387,413), pectin (U.S. Pat. No. 6,325,810), polycaprolactone (U.S. Pat. No. 6,337,198), polylactic acid (U.S. Pat. No. 6,267,776), polyglycolic acid (U.S. Pat. No. 5,576,881), polyhydroxyalkanoates (U.S. Pat. No. 6,245,537), dextrans (U.S. Pat. No. 5,902,800), polyanhydrides (U.S. Pat. No. 5,270,419), poly(vinyl pyrrolidone), and other biocompatible polymers. In some embodiments, the PEO has a molecular weight from, 400,000 to 2,000,000 g/mol. In some embodiments, the molecular weight of the PEO is about 900,000 g/mol. As contemplated by the present invention, two or more biocompatible polymers can be directly added to the aqueous solution simultaneously or sequentially.

In some embodiments, a silk solution and/or aqueous solution comprising silk protein has a concentration of about 0.1 to about 30 weight percent of silk protein. In some embodiments, the silk solution and/or aqueous solution comprising silk protein has a concentration of about 1 to about 20 weight percent of silk protein. In some embodiments, the silk solution and/or aqueous solution comprising silk protein has a concentration of about 1 to about 10 weight percent of silk protein. In some embodiments, the silk solution and/or aqueous solution comprising silk protein has a concentration of about 1 to about 5 weight percent of silk protein. In some embodiments, the silk solution and/or aqueous solution comprising silk protein has a concentration of about 5 to about 10 weight percent of silk protein.

In some embodiments, the film comprises from about 50 to about 99.99 parts by volume aqueous silk protein solution (e.g., from about 50 to about 95, from about 50 to 90, from about 50 to 85, from about 50 to 80, from about 50 to 75, from about 50 to 70, from about 50 to 65, from about 50 to 60, or from about 50 to 55 parts by volume) and from about 0.01 to about 50 parts by volume biocompatible polymer (e.g., from about 0.1 to 50, from about 0.5 to 50, from about 1 to 50, from about 5 to 50, from about 10 to 50, from about 20 to 50, from about 30 to 50, or from about 40 to 50 parts by volume).

According to various embodiments, a silk film may be from about 5 to about 300 μm thick. In some embodiments, a silk film may be between 5 and 250 μm thick, between 5 and 200 μm thick, between 5 and 150 μm thick, between 5 and 100 μm thick, between 10 and 200 μm thick, between 10 and 150 μm thick, or between 10 and 100 μm thick. Alternatively or additionally, thicker samples can easily be formed by using larger volumes or by depositing multiple layers.

Silk articles, such as silk films, may be made according to any method known in the art. An exemplary process for forming an article includes, for example, the steps of (a) preparing an aqueous silk fibroin solution comprising silk protein; (b) adding a biocompatible polymer to the aqueous solution; and (c) drying the mixture. In some embodiments, the biocompatible polymer is poly(ethylene oxide) (PEO). In some embodiments, the process for producing a silk article may further include step (d) of drawing or mono-axially stretching the resulting silk article to alter or enhance its mechanical properties. Additional methods of producing silk films may be found, inter alia, in U.S. Pat. No. 7,674,882, PCT application PCT/US2009/060135, and PCT application PCT/US2010/050698. According to various embodiments, silk films will be substantially uncrystallized prior to reflow.

In another aspect, the present invention provides methods for bonding a first silk fibroin layer with a second silk fibroin layer via a silk-silk interface including the steps of contacting a first silk fibroin layer with a second silk fibroin layer, and inducing reflow of silk fibroin of the first silk fibroin layer and silk fibroin of the second silk fibroin layer to generate a silk-silk interface with a bond strength of at least 500 kPa.

Adhering Silk Surfaces

The present invention provides, among other things, technologies for adhering amorphous silk surfaces to one another through induction of reflow at the silk surfaces (i.e., contact surfaces). According to various embodiments, the present invention provides technologies for achieving silk fibroin reflow within silk articles comprising at least one amorphous silk surface, such as, for example, silk films. As described below, it is contemplated that achieving reflow within a silk article (e.g., a first and second silk fibroin layer) will result in the formation of one or more bonds at the silk-silk interface. According to various embodiments, an amorphous silk surface is defined as susceptible to reflow when subjected to reflow conditions, such as, for example, temperature, pressure, and/or humidity, as described herein.

In this context, the glass transition temperature (T_{g}) of the protein is a parameter of particular relevance. As used herein, the term “reflow conditions” refers to a set of conditions wherein one or more amorphous silk surfaces is caused to be in a liquid-like state above its Tg, but has yet to reach a fully crystalized state. For example, in a silk film dried under ambient conditions, the water retained by the film acts as a plasticizer, significantly lowering the glass transition from 178° C. to ˜78° C. (X. Hu et al., Thermochimica Acta 2007, 461, 137-144). The actual T_{g} depends inversely on the water content and can be modeled as a function of the fractions of silk and water in the dried construct (N. Agarwal et al., Journal of Applied Polymer Science 1998, 63, 401-410). In order to achieve adhesion of amorphous silk surfaces via a reflow mechanism, various combinations of heat and pressure to a silk fibroin film are used. For any particular application or embodiment, reflow results from the use of a set of heat and pressure conditions sufficient to rapidly push the silk surface (e.g., a silk film) above its T_{g}, causing it to transition from a glassy state to a liquid-like rubber, allowing reflow of polymer on the nanoscale (J. J. Amsden et al., Advanced Materials 2010, 22, 1746-9).

In some embodiments, reflow of silk articles (e.g., comprising an amorphous silk surface) may be achieved using thermal induction of crystallization. As discussed herein, controlling the temperature and pressure applied during the reflow process affects the rate of water loss from, and energy addition to, the silk article (e.g., silk film), which in turn affects the molecular mobility and crystallization rate. These factors typically control the allowable time for thermal reflow, thereby affecting the properties of silk/silk interfaces. The control of these interfaces, afforded by controlling the parameters of time and temperature allow for additional silk fabrication options, expanding the role of silk films in the development of a range of devices.

In some embodiments, reflow of a silk article is achieved through exposure of the silk article to an elevated temperature for a period of time. In some embodiments, an elevated temperature is between 80° C. and 170° C. (e.g., between 80° C. and 150° C., between 80° C. and 120° C., between 80° C. and 110° C., between 80° C. and 100° C., between 80° C. and 90° C., between 85° C. and 120° C., between 85° C. and 110° C., between 85° C. and 100° C., and between 85° C. and 95° C.). In some embodiments, an elevated temperature is between 22° C. and 70° C. is the humidity is above 80 RH.

In some embodiments, a period of time is between 1 second and 1 minute (e.g., between 1 second and 50 seconds, between 1 second and 40 seconds, between 1 second and 30 seconds, between 1 second and 20 seconds, between 5 seconds and 1 minute, and between 5 seconds and 30 seconds). In some embodiments, a period of time is at least 1 second, at least 1 minute, at least 1 hour, or at least 1 day. In some embodiments, a period of time is between 1 and 24 hours, between 24 and 168 hours, between 1 week and 4 weeks, or between 1 month and 1 year.

According to various embodiments, reflow of silk articles may occur at an elevated or decreased pressure. Generally, it is contemplated that reflow of silk articles will be achieved more easily when there is a relative pressure difference being exerted on the silk article(s), such as one or more silk films. For example, when reflow of a first silk film with a second silk film is desired, it is contemplated that having either a positive or negative pressure differential between the first and second silk films is preferable to having no pressure differential exerted on the first and second silk films. In some embodiments, reflow is achieved at pressures between 0 and 1,000 pounds per square inch (PSI). In some embodiments, reflow is achieved at pressures between 10 and 1,000 psi, between 10 and 900 psi, between 10 and 800 psi, between 10 and 700 psi, between 10 and 600 psi, between 10 and 500 psi, between 10 and 400 psi, between 10 and 300 psi, between 10 and 200 psi, or between 10 and 100 psi. In some embodiments, reflow is achieved at pressures between 10 and 100 psi, between 20 and 100 psi, between 30 and 100 psi, between 40 and 100 psi, between 50 and 100 psi, between 10 and 90 psi, between 10 and 80 psi, between 10 and 70 psi, or between 10 and 60 psi. In some embodiments, reflow is achieved under vacuum conditions. In some embodiments, reflow is achieved at pressures at or above 1 psi, 5 psi, 10 psi, 20 psi, 30 psi, 40 psi, 50 psi, 100 psi, 200 psi, 300 psi, 400 psi, 500 psi, or 1,000 psi.

In some embodiments, reflow may be achieved or affected by the humidity present in the area or environment surrounding a silk article. Generally, an amorphous silk surface may have between about 10-12% water content, while a crystallized silk surface will have a water content between about 6-7%. In some embodiment, in order to achieve reflow, it is typically desired that the humidity of an area surrounding a silk article be sufficient to support a water content of between about 10-12% in the silk article during the reflow process.

According to various embodiments, achieving silk fibroin reflow within silk articles (e.g., a first and second silk fibroin layer), will result in a bond forming between the surfaces subject to reflow (i.e., at the silk-silk interface). In some embodiments, allowing reflow of silk films for a longer period of time results in increased bond strength at the silk-silk interface. In some embodiments, the silk-silk interface has a bond strength of at least 500 kPa, at least 750 kPa, at least 1,000 kPa, at least 1,250 kPa, at least 1,500 kPa, at least 1,750 kPa, at least 2,000 kPa, at least 2,250 kPa, or at least 2,500 kPa. In some embodiments, the silk-silk interface has a bond strength of more than 2,500 kPa.

As described above, reflow may be achieved in a silk article through thermal induction of crystallization. In some embodiments, provided methods replicate the rapid crystallization into β-pleated sheet-dominated structures observed in native silk fibroin dope by exposure to reflow conditions comprising heat, pressure, water vapor, and/or organic solvents. In addition to the effect on mechanical properties of a silk article, control of crystallinity is essential to the control of degradation of silk materials in vitro and in vivo. Mechanistically, the crystallization process is considered similar to that of synthetic block copolymers, occurring in three phases marked by microphase separation and micelle formation, crystal nucleation and growth, and crystal stabilization, in that order. Thus, the kinetics can be described adequately by Avrami analysis. This kinetic model yields characteristic sigmoidal-shaped transition curves. In order for crystallization to proceed, two requirements must be met: the material must be above its glass transition temperature (T_{g}) to allow sufficient chain mobility for the conformational transition, and sufficient activation energy must be supplied to initiate crystal nucleation. For both of these requirements, the water content in the material is an essential variable to control.

The effect of water on silk article T_{g} has been previously studied. Agarwal et al. found that controlling residual water in a dried silk film via relative humidity led to a decrease in T_{g} as water content increased, with the water acting as a plasticizer for the film. For films dried at ambient conditions (˜10.5% residual water), this results in a T_{g} close to ˜78° C. From this starting point, most current methods of crystallization remove water, (organic solvent) apply energy, (heat treatment), lower the glass transition via vacuum in a high relative humidity environment, (water vapor annealing) or a combination (heated water vapor annealing), to allow crystallization to occur.

For some of these mechanisms, removal of water may be an essential phase of the crystallization process. To this end, a modification to the model has been made by Strobl, and applied to silk, generating a four-phase model of crystallization. The remaining water in a silk film is divided into three classes, unbound freezing, bound freezing, and bound unfreezing waters, with increasing degree of association with the protein chains. In this model, the initiation of crystal nucleation and growth is predicated on removal of some of the unbound and bound freezing water from the film. In addition to crystallizing the film, it has been demonstrated that crystallinity, water content, and glass transition assert some level of control at film interfaces.

Pockets

In some embodiments, technologies provided herein are used to adhere silk articles (e.g., silk films) by inducing reflow and modulation of silk-silk interface properties so that adhered portions bound one or more unsealed regions, or pockets. In some embodiments, silk films are formed into multilayered compositions comprising a first silk fibroin layer and a second silk fibroin layer wherein at least a portion of the first and second silk fibroin layers are directly adhered to one another via a silk-silk interface such that the silk-silk interface defines a boundary around non-adhered portions of the first and second silk fibroin layers, thereby defining a pocket.

In some embodiments, provided multilayer compositions further comprise a third silk fibroin layer wherein at least a portion of the third silk fibroin layer is directly adhered to at least a portion of at least one of the first silk fibroin layer and second silk fibroin layer via a second silk-silk interface. In some embodiments, the second silk-silk interface defines a boundary around the non-adhered portions of at least one of the first and second silk fibroin layers, thereby defining a second pocket.

In some embodiments, provided multilayer compositions further comprise a third silk fibroin layer and a fourth silk fibroin layer, wherein at least a portion of the third silk fibroin layer is directly adhered to at least a portion of the fourth silk fibroin layer via an additional silk-silk interface to form an additional pocket. In some embodiments, the first silk fibroin layer and second silk fibroin layer are encapsulated within the additional pocket.

In some embodiments, such pockets may contain or be filled by a gas. In some embodiments, a gas may be or comprise air, nitrogen, argon, or CO₂.

In some embodiments, such pockets may contain or be filled by a liquid. In some embodiments, a liquid is an aqueous liquid or an organic liquid (e.g., an oil).

In some embodiments, such pockets may contain or be filled by one or more active agents, including for example one or more biologically active agents and/or one or more electrically active agents. In some embodiments, a biologically active agent is one or more bioactive compound such as an antibiotic, an antiviral, an antifungal, an anti-thrombotic, a fragrance, a vitamin, a nutrient, a food, a retroviral agent, a nanoparticle, a quantum dot, or a growth factor.

In some embodiments, such pockets may contain or be filled by a device (e.g., a degradable device).

It is specifically contemplated that, according to various embodiments, provided multilayer compositions may comprise a plurality of pockets formed from a plurality of silk fibroin layers.

Devices

In still another aspect, the present invention provides multilayered compositions including a first silk fibroin layer, a second silk fibroin layer, and a device, wherein at least a portion of the first silk fibroin layer is directly adhered to at least a portion of the second silk fibroin layer via a silk-silk interface and the device is located, at least in part, between the first silk fibroin layer and the second silk fibroin layer. In some embodiments, the device is located completely between the first and second silk fibroin layers. In some embodiments, the device is encapsulated within a pocket.

In some embodiments, provided silk articles and compositions comprising one or more pockets may be used to protect one or more devices, for example, a degradable device, from one or more environmental or other hazards (e.g., water). As used herein the term “degradable device” refers to a device that is meant to have a finite life span before being broken down or otherwise rendered non-functional. In some embodiments, a degradable device is biodegradable. In some embodiments, a degradable device may be a transient electronic device. As used herein, a “transient electronic device” refers to an electronic degradable device. In some embodiments, a transient electronic device is encapsulated within one or more pockets in a particular article or composition. Without wishing to be held to a particular theory, the use of silk fibroin as a protection material for degradable device, for example, transient electronic devices, could extend the lifetime of such devices by adding additional degradation control points while retaining biocompatibility and biodegradability, as well as adding further functionality, thereby expanding the role of this class of devices for implantable diagnostics and therapeutics. In some embodiments, the present invention also provides methods of making and/or using such compositions.

In some embodiments, provided compositions including one or more devices may be used as an implantable diagnostic and/or therapeutic tool. In some embodiments, suitable devices may comprise one or more of silicon (e.g., silicon membranes), titanium, platinum, gold, palladium, tungsten, iron, chromium, magnesium (e.g., magnesium conductors), and/or alloys thereof.

As described in the Examples below, in some embodiments, it is contemplated that a transient electronic device may be located within a pocket of a provided composition and that the silk fibroin layers which make up the pocket are themselves encapsulated in a pocket of a second provided composition (i.e., the device is effectively located within two pockets). In some embodiments, a transient electronic device may be effectively located in three, four, five, or more pockets using a similar structure. Also as described in the Examples below, such a layered structure may provide benefits including extension of the lifespan of a transient electronic device. In addition, the Examples below provide additional detail about the inclusion of a transient electronic device in provided compositions.

In some embodiments, a device (e.g., a transient electronic device) may be a sensor, a transmitter, antenna, transistor, any microelectronic component, optoelectronic components such as LEDs, VCSELs, integrated microlasers, and/or a receiver.

All patents and other publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

### EXAMPLES

### Example 1

### Interface Control of Semi-Crystalline Biopolymer Films Through Thermal Reflow

Silk Processing

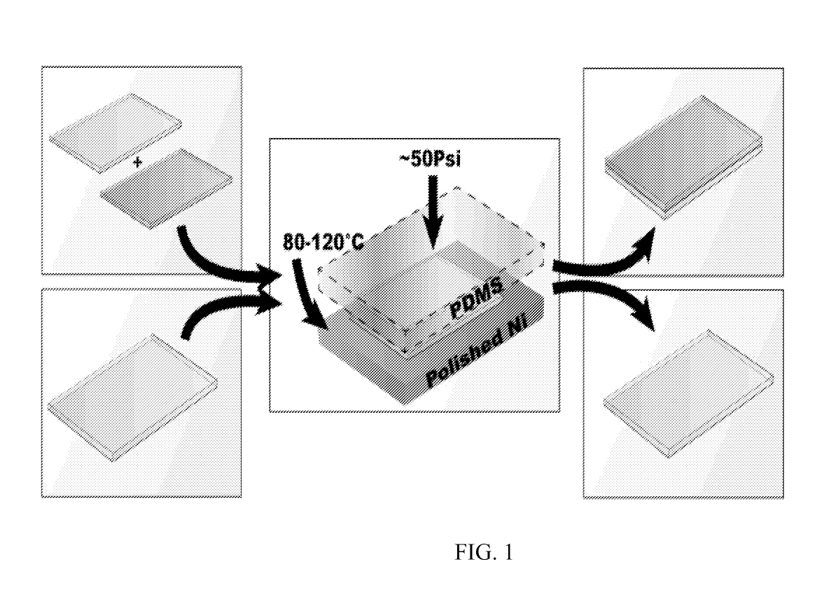

Regenerated silk solution was prepared via previously described methods. Briefly, cocoons of the silk worm B. mori were boiled in a 0.2 M solution of Na₂CO₃ for 30 min. to remove the sericin proteins. The dried fibroin bundles were then dissolved in a 9M solution of lithium bromide, and dialyzed against Milli-Q water for 72 h to remove the LiBr. This yielded ˜6% aqueous solution of silk fibroin, which was stored at 4° C. Films of ˜100 μm thickness were then cast on poly(dimethylsiloxane) (Sylgard 184, Dow Corning Corp., Midland, Mich.) substrates and dried at ambient conditions (˜23° C., ˜25% relative humidity). The films were processed as shown in FIG. 1. One or two dried films were placed between a polished nickel substrate of ˜500 μm thickness for even heating, and a PDMS over-layer of ˜3 mm thickness for even pressure, and heated from the bottom to temperatures ranging from 80-120° C., for times ranging from 5-60 seconds. Concurrently, ˜50 Psi of pressure was applied from the top via freestanding weights. One film was pressed with a patterned nickel substrate for imprinting experiments, and two films were pressed together for adhesion testing.

Thermal Imaging

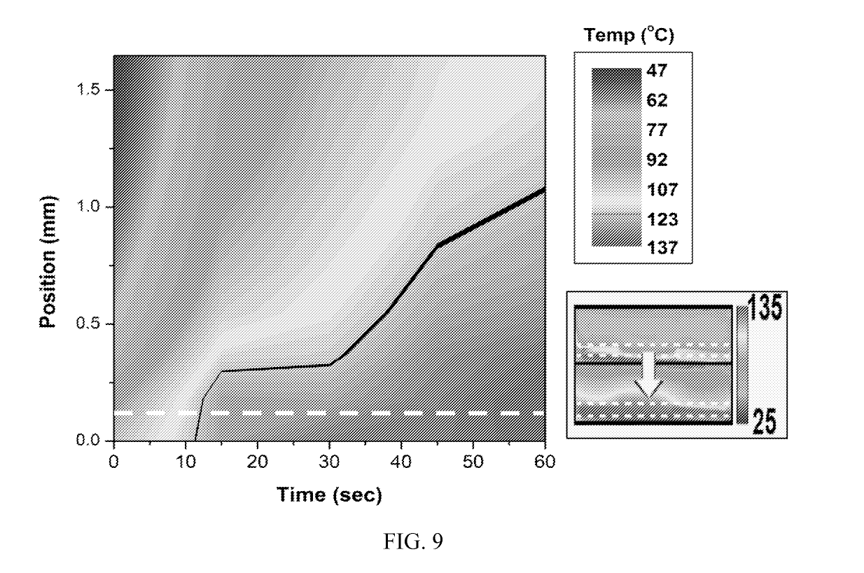

Thermal imaging of the silk films during heating was carried out via an infrared camera (FLIR SC600, FLIR Systems, Boston, Mass.). Imaging of the silk and PDMS sheets were acquired as they were heated from ambient to steady-state heated temperatures. A ˜1 frame/second video was taken over the course of 60 seconds during the heating process. The image section corresponding to the silk film was analyzed to develop heating curves for the silk material alone. Bulk silk heating curves as a function of time were used for analysis after determining that the temperature within the film was consistent (FIG. 9).

For all analysis work, unless otherwise specified, the spatial temperature within a film as was considered to be at a steady state for the duration of the processing. This is a reasonable assumption given FIG. 9, which shows heating curves for the process, generated from thermal images of a ˜1.6 mm thick silk block heated over 60 seconds by the same process. The primary plot in the figure shows heating over time, with positions corresponding to the polished Ni interface at 0.0 mm, and the PDMS interface at 1.6 mm. The small inset on the bottom right shows the raw IR images, with tops and bottoms of the silk films marked by the dashed lines. The figure shows that for the experiments conducted, the silk has heated to ˜100° C. nearly instantaneously, and has reached a minimum of the set temperature by about 10 seconds. The film also continues to heat above the set temperature as heat continues to flow into the PDMS above the silk film. Thus the set temperature is only an estimate, and refers more aptly to the rate of heat flow through the film. Without wishing to be held to a particular theory, it is possible that the temperature within the film is independent of height away from the heat source given the small thicknesses in question

Thermal Gravimetric Analysis

Residual water content of the films was assessed after varying treatment times and temperatures via thermal gravimetric analysis (TGA) (TA instruments Q500 New Castle, Del.). Films were heated to 200° C. at a rate of 20° C./min, and monitored for mass loss, which was calculated as the difference in mass over the course of heating divided by the initial mass, according to established procedures.

Analysis of Silk Secondary Structure

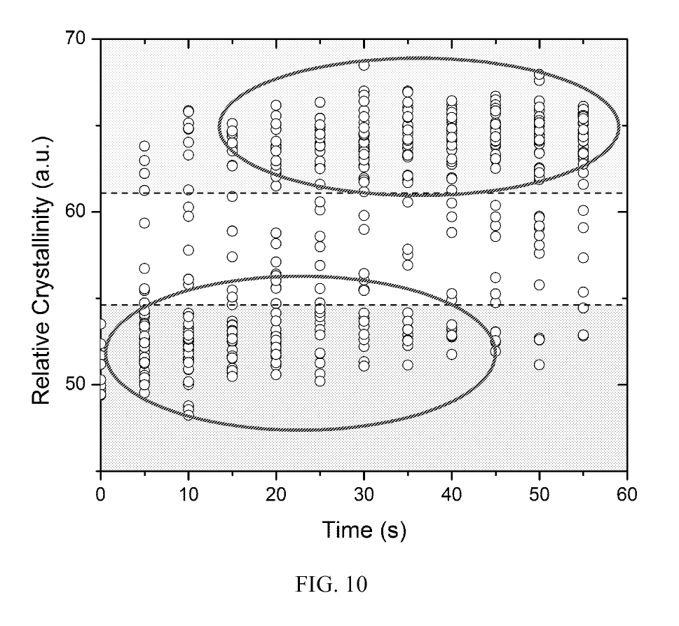

β-sheet crystallinity of treated films was determined by analysis of the Amide III band of the Fourier-transform infrared (FTIR) (Jasco FTIR 6200, Jasco Inc., Easton, Md.) spectra of the films. Sample films were pressed for 5-60 seconds in 5 second intervals at temperatures of 80, 85, 90, 95, 105, and 120° C. The spectra were collected using an attenuated total reflection (ATR) detector, on which 50 scans were co-added per collected spectrum. At the same time, a cosine apodization was applied by the software. The amide III band (1200-1350 cm⁻¹) was then analyzed for secondary structure via curve fitting. Often, the amide I band (1600-1705 cm⁻¹) is used to quantify protein secondary structure, but due to the large absorbance of water near 1650 cm⁻¹, this was avoided, as the water content of the measured films varies. To curve fit, the spectra were normalized, and then fit to 12 overlapping Gaussian bands via a Levenberg-Marquardt function in OriginLab 8.6, similar to established methods. The bands were identified according to the work of Xie and Liu, and the total β-sheet contribution was estimated. These values were taken to be estimates of the relative crystallinity, and were compared to quantifications of both untreated (˜50%), and methanol crystallized (˜61%) films. The films were further divided into crystallized and uncrystallized bands by analysis of the raw quantified data (see FIG. 10).

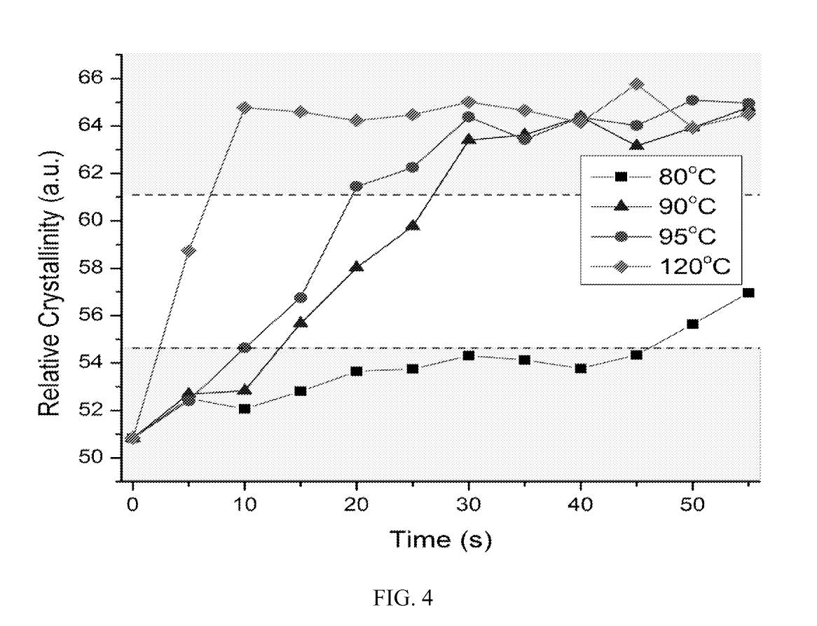

FIG. 10 shows the results of quantified FTIR scans, representing relative β-sheet content, without respect to temperature. As the red circles highlight, the density of the measurements was grouped into two clusters, which overlap slightly at middle times. Very few points lie between these clusters, which indicates that the crystallization process occurs primarily in an all or nothing fashion. While at lower temperatures an equilibrium may be reached below this threshold, for most of the measured temperatures this was not found to be the case. Based on this, the median crystallinity value of each time point was calculated. As expected, there was a large gap in the median over time, between the values of 54% and 61%. Therefore, everything below 54% was considered to be uncrystalized, and everything above 61% was considered to be crystallized as indicated by the dashed lines in FIG. 4. This matched well with the results for untreated and methanol treated films reported herein.

Analysis of Silk Film Reflow

Pre-treated films were subsequently imprinted to examine residual reflow potential. Films were pre-treated in the following groups: as-cast, 95° C. 5 seconds, 95° C. 25 seconds, 120° C. 5 seconds, 120° C. 10 seconds, and 120° C. 60 seconds, and then imprinted with a 3600 grooves/mm diffraction grating at 105° C. for 30 seconds, via an established procedure. Atomic force microscopy (AFM) (Veeco Dimension 3000, Bruker Inc., Santa Barbara, Calif.) was utilized to measure the topology, and the results were compared. Both the hole depth and change in root-mean-square (RMS) roughness characteristic (Rq) of the grating were calculated.

Mechanical Testing

Films were pre-treated to the same thermal conditions as used in previous experiments. Next, they were overlapped to an area of ˜20 mm² in a typical lap shear geometry, and heat-treated again at the interface to bind the films. The interfacial strength was tested by applying a tensile force parallel to the interface, following ASTM D3136, with minor changes made due to sample geometry limitations. The breaking force was area normalized to calculate the interfacial strength.

Microscopy

Films of differing initial crystallinity and water content were processed at 105° C. for 30 seconds. The films were then split with a razor blade so the cross sections could be analyzed. SEM measurements were performed on a Zeiss EVO MA10 (Carl Zeiss Microscopy, Ltd., Cambridge, UK). For optical measurements (Olympus IX71, same issues), one of the two laminated layers was doped with melanin to provide contrast. To form these, synthetic eumelanin (CAS 8049-97-6, Sigma Aldrich, St. Louis, Mo.) was dissolved in a ˜pH 11.5 solution of NaOH, by gentile heating and sonication. This produced a brown solution with continuous melanin dispersion. The pH of the solution was then adjusted to pH 7, yielding an ˜1% solution of eumelanin in water. The melanin solution was mixed into the previously described 6% aqueous silk solution during casting, and the volume casted was adjusted for the decrease in silk concentration, to ensure equivalent film thickness.

Statistical Analysis

All Experiments were carried out in replicates of three, with the exception of the FTIR analysis, for which n=9. Mean and standard deviation were calculated for all analyses, and unless otherwise noted, data are presented as mean plus/minus standard deviation. A one-way ANOVA (p<0.05) was used to verify trends where appropriate.

### Results

Heat Transfer, Water Content and Crystallinity

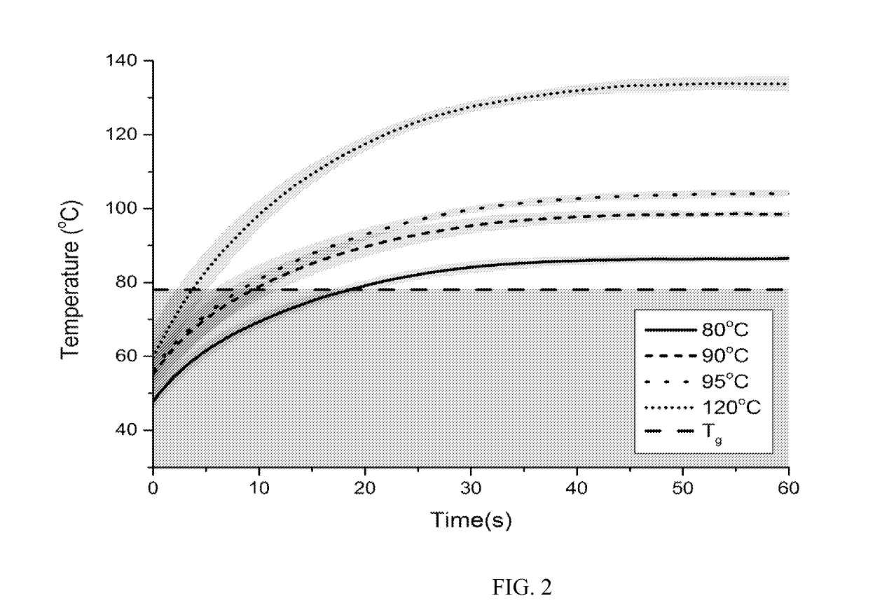

As an initial step in understanding the reflow mechanism, and its effect on silk interfaces, the primary physical processes occurring during the imprint/lamination process were quantified. Heating curves for the films based on the reported temperatures of the experimental setup were first generated (FIG. 2). In all cases, the rise time to steady state was dependent on the temperature, and the steady state value was higher than the set temperature. This may be due to continued heat flow into the PDMS, which has a low (˜0.15 W/m*K) thermal conductivity³⁴ and based on the placement of the feedback thermocouple in the heating system. The dashed line on the plot represents the glass transition temperature for ambient dried films. Thus, crystallization of a film pressed at these temperatures cannot occur prior to this point. Times at which the T_{g} was reached for different temperature treatments are reported in Table 1 below:

TABLE 1
Time to reach thresholds of conformation shift, and potential influences
on reflow time, as measured by crystallization plateau time minus
bound water onset time (all values are in seconds)

| | 80° C. | 90° C. | 95° C. | 120° C. |
|---|---|---|---|---|
| Tg | 45 | 8 | 4 | 2 |
| Bound Water | ∞ | 20 | 15 | 0 |
| Onset | | | | |
| Crystallization | 45 | 12 | 10 | 2 |
| Onset | | | | |
| Crystallization | N/A | 28 | 20 | 6 |
| Plateau | | | | |
| Reflow Time | 0 | 8 | 5 | 6 |

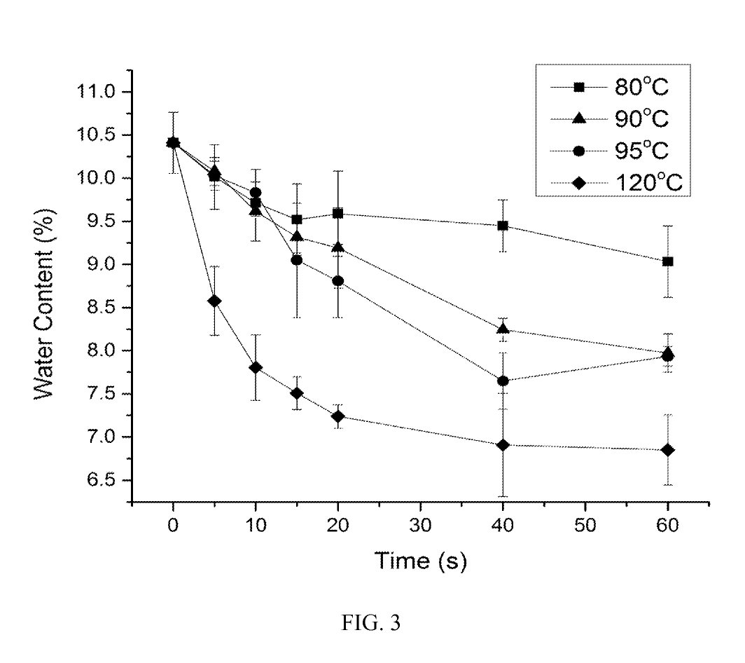

The rate of water loss tended to increase with increasing temperature (FIG. 3), in a manner analogous to the heating curves shown in FIG. 2. Initially, ˜10.5% of the mass of the films consisted of water, but 30% of that water (˜3% of the initial mass) was lost during the pressing process. The removal of water allows for further molecular scale motion of the silk protein chains. Based on the Strobl model, the initial burst of water loss seen here could be due to the release of the free freezing, or unbound water, followed by the secondary release of the loosely associated freezing bound water. The strongly associated non-freezing bound water would then make up the remaining 7% water. Based on these results, the time at which the freezing bound water begins to be released has been estimated and is reported in Table 1.

The rates of crystallization for each treatment temperature were determined from the FTIR analysis (FIG. 4). The thresholds pertaining to uncrystallized and crystalized samples are shown in gray. As expected from both the heat and mass transfer curves discussed above the rates were found to be faster for higher temperature treatments. For the low temperature conditions, the characteristic sigmoidal shape can be seen, as would be expected given the four-phase model for silk film crystallization. Interestingly, for almost all conditions, there is no difference in the apparent plateau at ˜64%. Intersection of each curve with the lower threshold can give an estimate of the onset time of the rapid crystal growth phase (Table 1).

Reflow Control

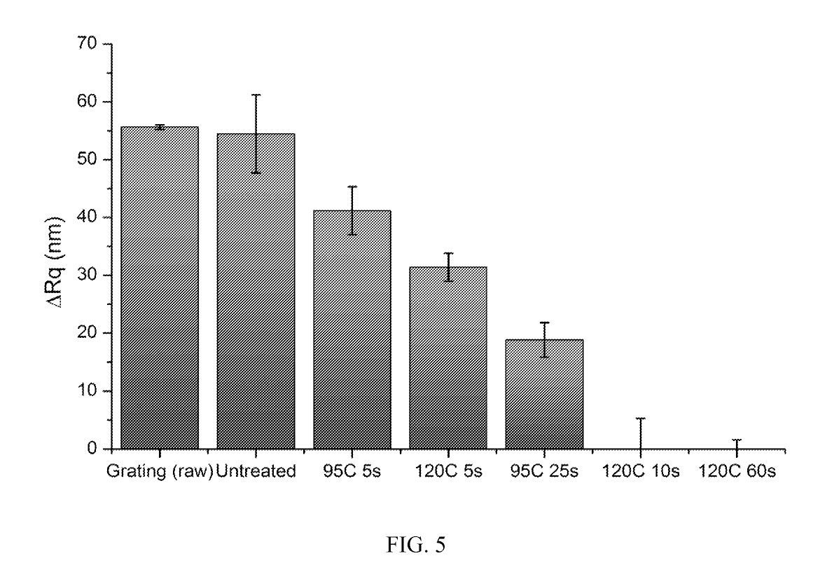

The characteristics of reflow as a consequence of thermal treatment has not been characterized or controlled, although it has been suggested by previous findings on thermal imprinting techniques. To gain further insight into the scale on which reflow in silk films can occur and determine control points for this parameter, imprinting results for films of varying thermal histories were quantified (FIG. 5). RMS roughness (Rq) of the pre-processed imprinted films was quantified and a clear trend of degree of replication, which diminishes with increasing treatment, was found. While the untreated condition replicated the surface near flawlessly, the fully crystallized film did not reflow at all, leaving no imprint on the surface.

Silk/Silk Interfaces

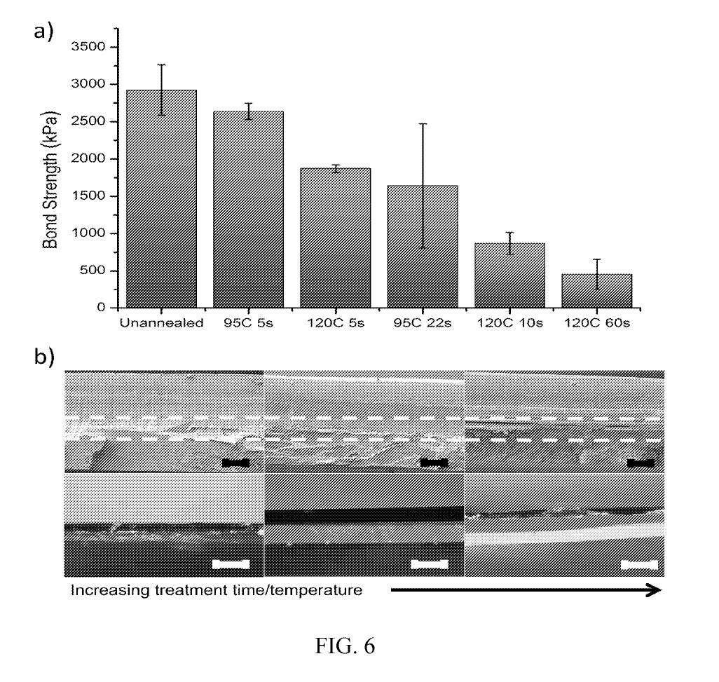

Results of the bond strength analyses are shown in FIG. 6a. As hypothesized, there was a decrease in bond strength with decreasing reflow time. The strength, initially measured to be nearly 3 MPa for samples without pretreatment, decreased to less than 500 kPa as the reflow times decreased. The interface between laminated layers varied as function of allowable reflow as shown in the SEM and optical micrographs (FIG. 6b). In the top set of images, the SEMs show no discernable gap at the interface, suggesting intermixing of the two layers. The larger scale optical image indicates that such intermixing, if occurring, happens on a microscale, as mixing of the melanin-doped silk into the transparent film near the interface is not visible. Differences begin to emerge in the second two conditions. As β-sheet content increased and reflow time decreased, evidence of weak adhesion begins to appear at the interface, both on the macro- and microscale levels. These weak spots would likely lead to a decrease in interfacial strength, as well as some variation depending on location. Therefore, if the intermixing and filling of nanoscale features in laminated films is indeed a function of reflow, this should influence the bond strength between the two layers.

The experimental results on silk films presented above have interesting implications for the behavior of semi-crystalline biopolymer interfaces. Given that the results indicate reflow as a control point for silk/silk interfaces, its relationship to controllable physical parameters such as temperature and time should be evaluated in order to correspondingly control the interface properties. The important physical thresholds relating to these parameters are presented in the first four rows of Table 1, as discussed above. Based on the times tabulated in the table, some predictions about the behavior of imprinted/laminated films can be made. As expected, the crystallization onset always occurs after the glass transition temperature has been reached. This temperature indicates that enough energy is available for molecular rearrangement. However, reflow may also depend on the onset of bound water release/mobilization. This effect is due to the breaking of the loose association of the bound freezing water that occurs following this plateau. Without wishing to be held to a particular theory, this may be the onset point for nanoscale and macroscale reflow that may occur in the films. The crystallization plateau represents the point at which the new conformation of the film has been locked into place, and the water content is no longer changing. At this point reflow should no longer be possible. The last row in Table 1 represents the times for reflow as determined by this hypothesis, calculated as the difference between the time to reach the crystallization plateau, from the FTIR analysis, and the time at which bound water begins to leave the film, from the TGA analysis. This, we posit, represents the thermal reflow potential of ambiently dried silk fibroin films. If the basic parameters of the process can be used to induce and control reflow along these lines, the interfacial properties of the fibroin films can therefore be controlled.

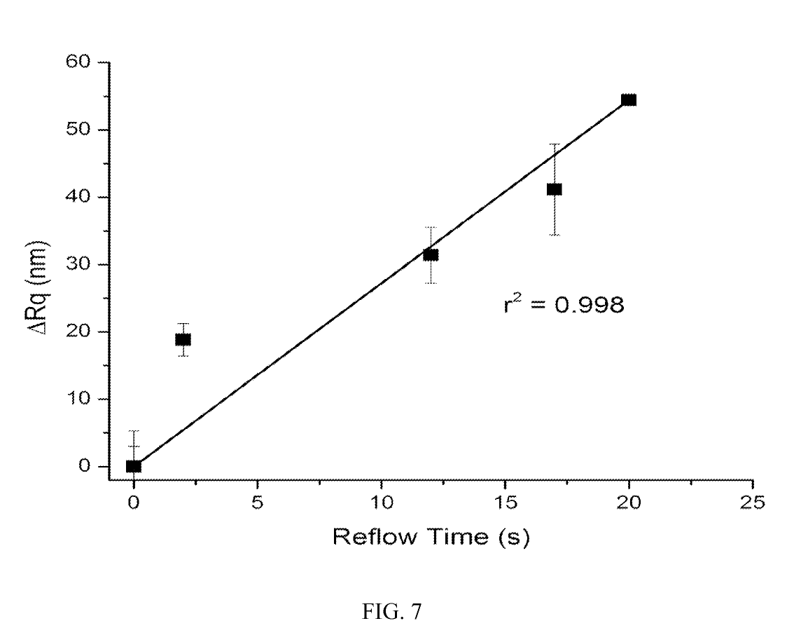

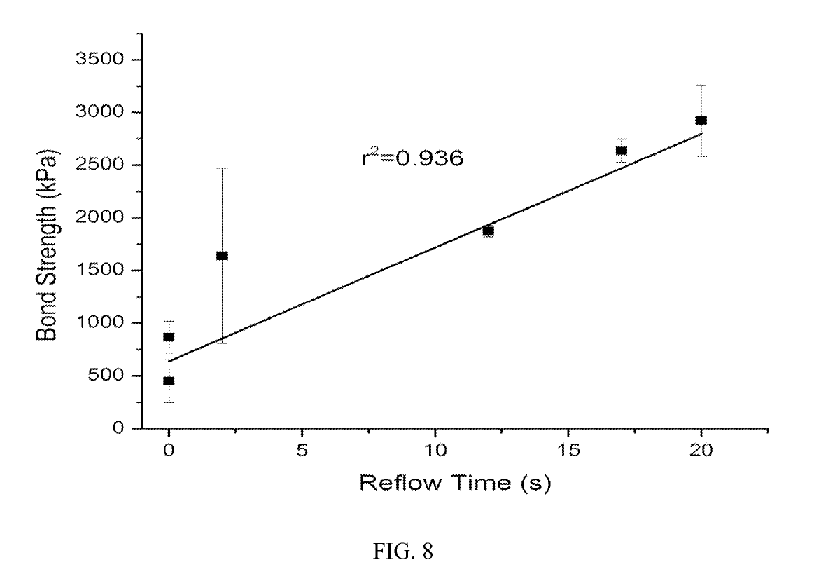

To analyze this hypothesis in light of the collected data, reflow times were estimated for each treatment condition in the nanoscale reflow and mechanical testing experiments. This was done based on the data in FIG. 5 for nanoscale reflow, and FIG. 6a for mechanical testing. Given that only higher temperatures were used, the crystallinity induced by the pretreatment was used as a starting point, and then compared to the plateau point. The difference in time between these crystallinities at 105° C. was considered to be an estimate of the reflow time. Rq was then plotted against reflow time (FIG. 7) and the same method was used to plot reflow time against bond strength (FIG. 8).

The results show a clear linear trend, where increasing reflow time marks better replication. A linear fit of the data (with an r² value of 0.998 for the nanoimprinting experiment, and 0.936 for the bond strength), further supports the trend. The only non-conforming point (within experimental error) was noted for the case of 95° C., 5 second pre-treatment. While not wishing to be held to a particular theory, this is likely due to residual unbound water in the silk film, which was not accounted for in the reflow time calculation, where the only factor considered was crystallinity. However, for all other cases, the thermal energy provided is such that most unbound water has been removed, eliminating this as a factor. Thus, the data suggest that reflow in silk films is indeed primarily a function of crystallinity, although water content is an important secondary factor. These can both be controlled by adjusting process parameters such as treatment time and temperature. Furthermore, these data demonstrate that thermal control of reflow in silk films can be leveraged to control silk/silk interfaces through varying degrees of film intermixing as well as by conformal filling of the nanoscale features in adjacent films.

This proposed adhesion control has further implications on the physical mechanism of adhesion at silk/silk interfaces. The existence of a clear linear trend with reflow suggests that mechanism of adhesion is dominated by intermixing, with little chemical cross-linking occurring between the layers. This is opposed to the hypothesized adhesion mechanism of similar polymers such as polyimides, which behave similarly on a macroscale, but are thought to have a cross-link-dependent adhesion. Furthermore, an intermixing dominated adhesion mechanism suggests that parameters such as surface roughness, or additional cross-linkers, could be used to further strengthen the silk/silk bond at the interface.

Control of silk/silk interfaces through the reflow mechanism described herein could have interesting applications in the fabrication of devices in two key areas. Rapid nanoimprinting has already been demonstrated in our previous work. However, based on the new information, the nanoimprinting mechanism could be extended to the use of pre-crystallized silk films as imprinting masters, creating a new protein-protein imprinting method. Such a method would lessen the dependence on the currently used metal and silicon based masters, which are both expensive and difficult to produce, especially for structures optically resonant in the visible regime. Furthermore, a flexible silk master would allow for the possibility of conformal imprinting on non-planar surfaces, which is currently difficult to achieve by all available nanoimprint lithography methods.

If, on the other hand, the silk/silk interfaces were tailored to maximize interfacial strength, lamination based techniques could be used to build multilayer structures, which were not possible using the silk fabrication toolbox prior to the present invention. This would open up new avenues in the use of biocompatible, resorbable fibroin layers for the passivation and protection of implantable devices. Additionally, dopants or other modification could make the fibroin layers active themselves, creating new possibilities for optical and electronic multilayer devices. Further, the absence of formation of chemical crosslinks during the processing described leaves options for relatively simple and green chemistry reuse of the materials to reform new masters to supplement the utility of this technology.

This Example shows, among other things, that through application of heat and pressure, the mass transfer of water from the film and rates of crystallization can be controlled. The coupling of these factors offers compelling methods for rate control of silk film reflow, which can in turn be used to control silk/silk interfaces, and suggests an intermixing dominated adhesion mechanism. Control of these interfaces has been used already for nanoscale imprinting of silk films, which has been extended to an all-silk “protein-protein imprinting” method. Fabrication of multilayer silk devices was enabled by this mechanism, enhancing the standing of silk as a platform for the development of devices that further bring the principles of high technology into biomedical applications.

### Example 2

### Rapid Lamination of Multilayer Silk Fibroin Pockets for Controlled Degradation and Enhancement of Function of Transient Electronic Devices

In this Example, use of certain provided methods are described to provide an indirect encapsulation strategy for transient devices with silk fibroin. This Example also demonstrates how such an encapsulation strategy could be used to allow for additional control of device degradation as well as enhancement of device functionality.

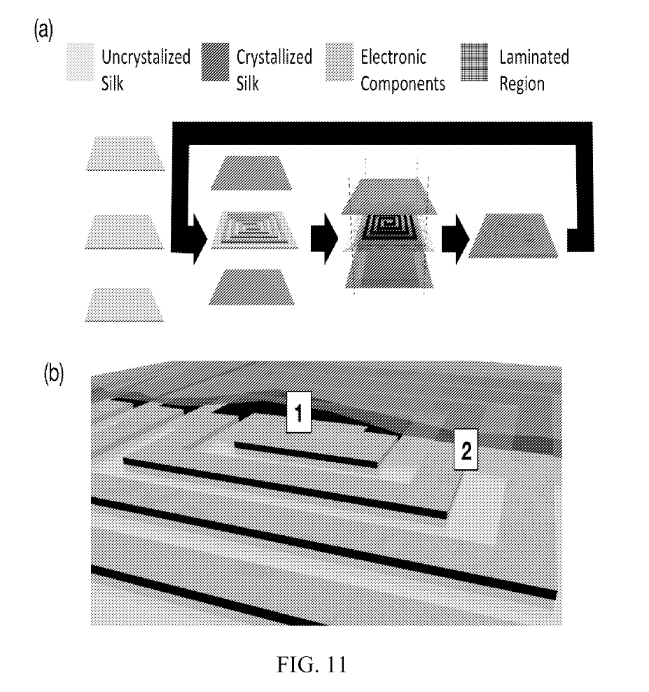

A scheme of the provided method used in this Example is shown in FIG. 11a. Briefly, transient electronics fabricated by existing techniques on a silk substrate are sandwiched between two treated films of varying crystalline and diffusional properties. Sealing the outside of these films creates a small air pocket, which will provide protection to the water sensitive components of the device. Additional protective layers can be added by repeating the process with the fabricated pocket in lieu of a bare device. After exposure to a wet environment, rapid swelling of the silk increases the effective volume and collapses the air pocket, thus initiating device degradation. This produces two possible interfaces, a silk/air/device interface and a silk/device interface, as shown in FIG. 11b. The properties of the protective films and number of layers thereby determine the transience time of the device, through spatial control of these interfaces. Such a protection strategy will prevent degradation of the fragile transient components during encapsulation, as well as uncouple the fabrication of the device and protecting pocket, allowing for additional elements such as dopants and structural elements to be considered in the encapsulation.

### Experimental Methods

Silk Processing

Films to be laminated were cast from regenerated aqueous Bombyx mori silk fibroin solution, production of which has been previously described (see Rockwood et al., Materials fabrication from Bombyx mori silk fibroin, Nat. Protoc., 2001, 6(10):1612). Briefly, B. mori cocoons were boiled in a 0.02M aqueous solution of sodium carbonate for 10 minutes to remove the immunogenic sericin protein, which acts as a glue holding the fibroin filaments together. The remaining fibroin was then rinsed thoroughly in deionized (DI) water and allowed to dry overnight. Next, the fibroin was dissolved in a 9.3M aqueous solution of lithium bromide at 60° C. for three hours. The lithium bromide was then removed from the solution via osmotic stress. The solution was placed into dialysis cassettes (Slide-a-Lyzer, Pierce, MWCO 3.5K) and dialyzed against water for 36 hours. The resulting 5-8% (w/v) aqueous solution was purified through centrifugation prior to casting. Finally, the silk films are cast onto PDMS substrates at 1 mL/in² and allowed to dry under ambient conditions, to produce films of ˜85 μm thickness.

Silk Film Treatments

Water vapor annealing: The films were placed in a vacuum oven with a 400 mL container of DI water. The vacuum was set to −80 in Hg, and the chamber was sealed for 24 hours to treat the films by water vapor exposure.

Heat treatment: The films were placed in between a nickel shim and a 5 mm thick PDMS overlayer, and heated rapidly to 120° C. for 30 seconds, while 135 Psi of pressure was applied from the top.

PVP 66% films: PVP 66% films were prepared by thoroughly mixing 7% aqueous silk fibroin solution with a 7% aqueous solution of Poly(vinyl pryrrolidone) (PVP K90), (MW 360 kDa) (CAS 9003-39-8, Sigma Aldrich, St. Louis, Mo.) in a 2:1 (PVP:Silk) ratio. Films of the resulting solution were cast on PDMS substrates at 1.5 mL/sq. in. and dried under a small fan over the course of 3 h before being removed. The films were then placed in 100% Methanol for 30 minutes, crystallizing the silk, and removing the methanol-soluble PVP.

Methanol treatment: The films were then placed in 100% methanol for 30 minutes to crystallize them.

Mechanical Testing

Two 85 μm films were overlapped in a modified lap-shear geometry (similar to ASTM D3136), with 1 cm by 1.5 cm silk films of 100 μm thickness overlapped by 1.5 mm to produce an adhesion area of 150 mm². The films were pre-treated using the previously described cross-section of available silk film beta-sheet treatments and laminated with and without a 30 μm silk adhesive layer in between at 120° C. and 135 Psi for 30 seconds. The films were then tested in tension (Instron 3360, Instron Inc.) to failure, and the results were normalized to determine the bond strength of the samples.

Multilayer Membrane Evaporation

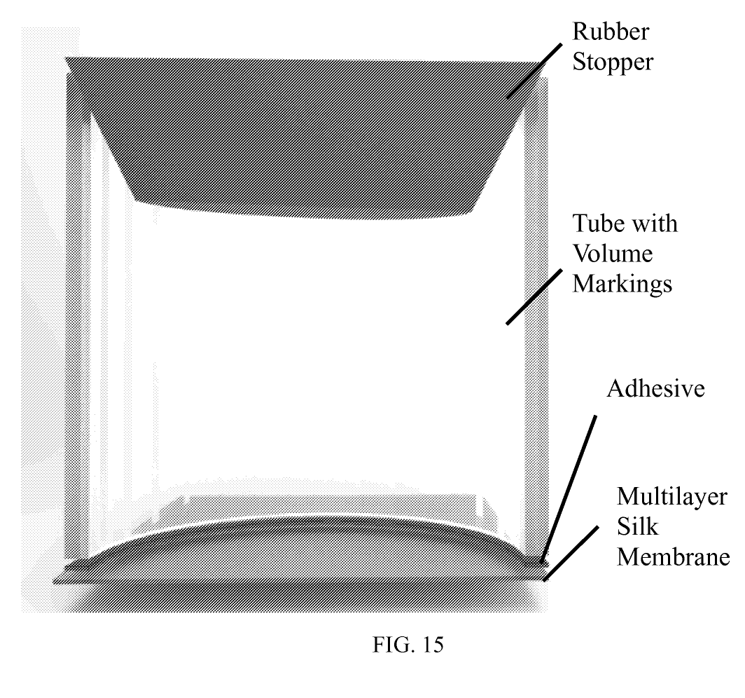

Multilayer membranes were fabricated by lamination for membranes of 1, 2, 3, 4 and 5 layers. To keep the membrane thicknesses comparable, individual layers of increased layer samples were cast at reduced volumes for a total cast density of 1 mL/in² of 7% aqueous silk solution. The individual layers were then crystallized by heat treatment. In between each layer, a 30 μm uncrystallized adhesive layer was stacked. Prior to stacking, a 35 mm diameter biopsy punch was used to remove the center of the adhesive layer. The stacks were then laminated together at 120° C. and 250 Psi for 30 seconds, with pressure applied only to the outer glue containing portion of the films. These membranes were then hydrated for 30 minutes and affixed to the bottom of 35 mm diameter tubes with commercial adhesive (Loc-Tite 406, Henkel Ltd, UK), aligning the region of air interface in the membrane with the inside of the tube (FIG. 15). The tubes were then filled with 5 mL of DI water, and the tops were sealed to prevent evaporation.

Unless otherwise stated, the samples were exposed to ambient conditions for two weeks. Each day the volume remaining in the tube was measured. The water loss curves were fit to a linear function by a least-squares regression algorrithm (Origin 8.5, OriginLab Inc.) to determine the mean leak rate for each sample.

Magnesium Resistor Degradation

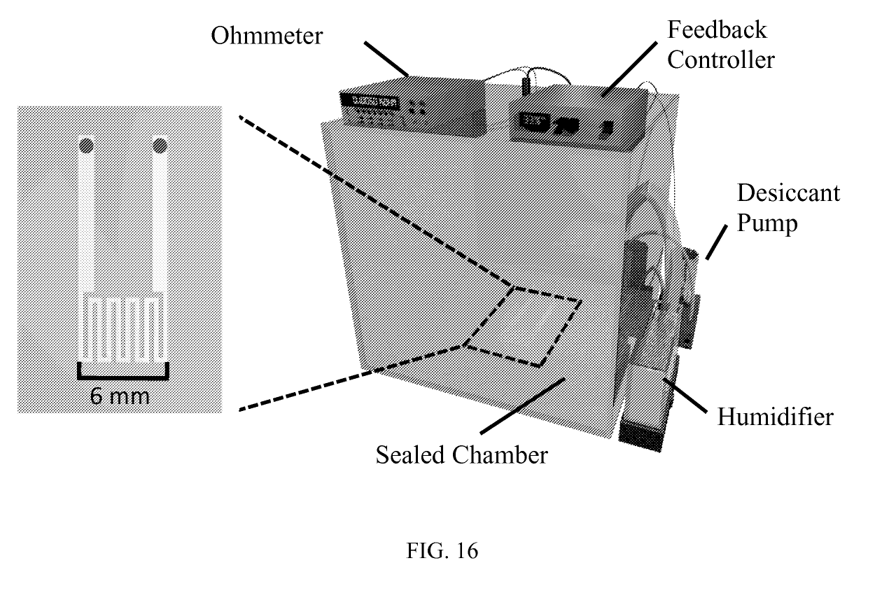

Magnesium resistors were deposited on clean glass slides through a 12.5 μm polyimide shadow mask by electron beam evaporation. A 15 nm titanium adhesion layer was first deposited below 300 nm Mg. The Magnesium trace resistors were then placed in a custom built acrylic chamber with a feedback controlled humidity regulation system installed (Model 5100, electro tech systems inc.). The resistance of the trace was probed at 2 minute intervals with a digital multimeter (Keithley 2700, Keithley inc.) for the duration of resistor degradation in either 90% relative humidity or with direct application of 500 μL DI water (FIG. 16). Resistances were normalized to their initial value to account for variability in fabrication, and the resulting time curves were fit using the existing analytical model for reactive diffusion.

In Situ Device Degradation

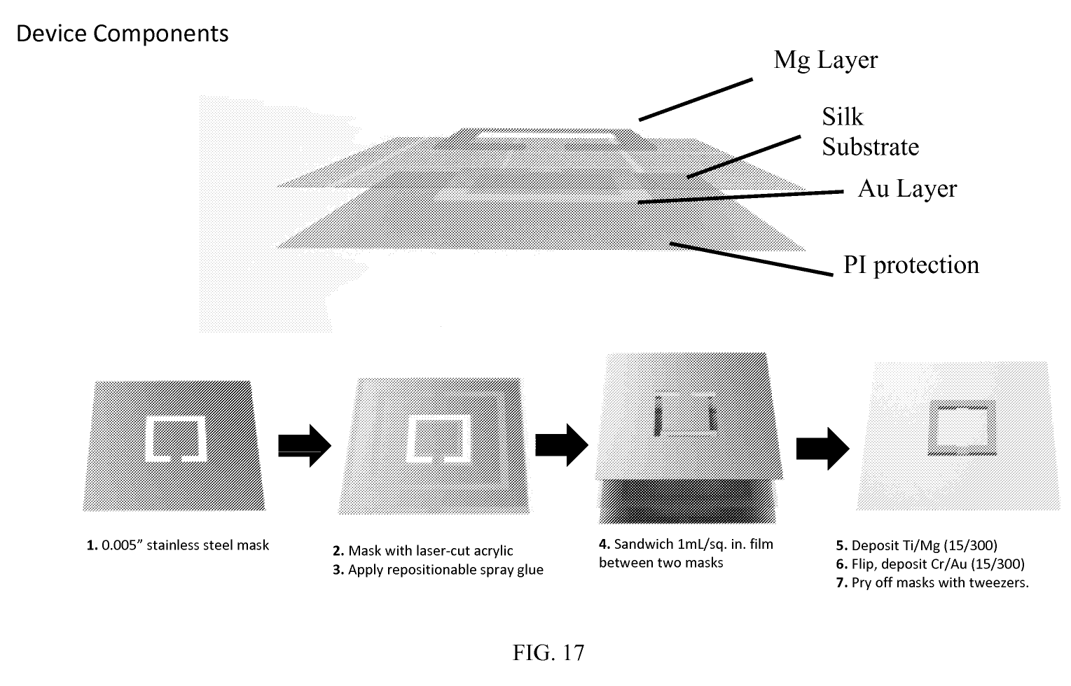

In this Example, a bilayer design was utilized for the antennas, consisting of a simple square split ring resonator with 8 mm unit cell length on each side of an 80 um thick silk film, with the resonator gaps in opposing directions. The top-side resonator consisted of 600 nm of Mg with a 30 nm thick Ti adhesion layer deposited by electron beam lithography, while the bottom resonator consisted of 400 nm of Au with a 20 nm thick Ti adhesion layer deposited by the same method. Below the gold a 15 um thick polyimide tape was used to protect the gold and limit loss of signal due to buckling of the substrate for the purposes of the test (FIG. 17). These devices were encapsulated in 0, 1, 2, or 3 silk pockets by the method described previously, using 1 mL/in² silk protection layers and 0.5 mL/in² adhesive layers. Also tested was a single layer pocket in which the silk protection layers were of equivalent thickness to the total three layer system. This pocket was fabricated by lamination of three 1 mL/in² uncrystallized silk films together to produce the crystallized protective layers, which were in turn used to fabricate samples. The pockets were affixed with commercial adhesive (Super 77, 3M inc.) to an acrylic well to contain the water exposure to the top-side of the pocket, thereby limiting water underneath the device from obscuring the signal. The devices were placed directly on top of a transceiver antenna fabricated on PCB and attached to a network analyzer. During the experiment, 1 mL of DI water was added to the well and the resonant response of the encapsulated antenna was monitored at one-minute intervals until the signal was lost. The resonant peaks were fit to lorentzian functions for each case, and the antenna quality factor (Q) was calculated as Q=fₒ/FWHM for the fitted parameters. The Q factors were normalized to their initial values in each case to account for differences in response due to minor variations in sample setup and defects in fabrication. To further analyze the behavior, the onset time of the rapid phase of degradation was determined for each sample by identifying the point at which degradation exceeded 3% per minute.

Antibiotic Pockets

In this Example, in order to test for the ability of provided pockets to protect a transient electronic device in a biological system and reduce or prevent infection, such as infection caused by introduction of the pocket, a 7% silk solution was mixed with Ampicillin sodium salt (CAS 69-52-3, Sigma-Aldrich) at 1 mg/mL, and films were cast from the resulting mixture onto PDMS molds at 1 mL/sq. in. and allowed to dry at ambient conditions for 24 hours. Silk films without antibiotic were cast at the same time as a control. The films were stored at 4° C. when not in use. The dried films were cut into 1 cm by 1 cm squares, and sealed on the edges at 120° C. for 5 seconds, with 150 Psi of pressure. E. coli were grown in liquid culture with Tryptic Soy Broth for 8 hours, and then plated onto Tryptic Soy Agar. Bacterial lawns were then treated for 30 minutes in one of four groups: silk pocket, silk pocket+antibiotic, 1 mL antibiotic only, and nothing. Lawns were allowed to develop overnight, and the zone of inhibition was measured after ˜18 h of growth, using Image J software.

### Results

Lamination Method

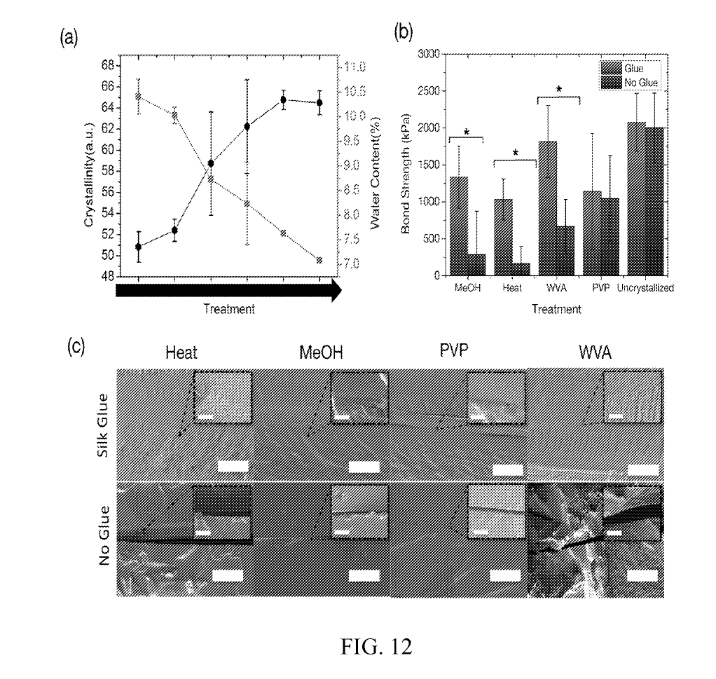

Effectiveness of the pocket strategy for encapsulation requires adequate sealing of the edges of the pocket. In this Example, we introduce multilayer silk film lamination as a method for this purpose, following the scheme shown in FIG. 11a. The materials to be laminated are arranged as desired, before being rapidly heated to 120° C. and pressed together with 135 Psi of pressure for 30 seconds. This process causes a rapid efflux of water from the film, coupled with an increase in beta-sheet crystallinity of the silk, as shown in FIG. 12a. During this time, intermixing of the two silk layers can occur by a reflow dominated mechanism discussed in detail elsewhere herein, leading to a weld at the interface. The potential for intermixing of the layers is dependent on their current crystalline state and water content. Untreated, wet layers have a high reflow potential, which does not exist in dry pre-crystallized films as will be used in fabricating pockets. However, we addressed this issue by adding a third ˜30 μm thick untreated silk film that was placed in the area of overlap between the films to be laminated, to act as an adhesive. Here, reflow of the central film into the two crystallized outer films initiates the bond, which is less dependent on the initial crystallinity of the films.

In this Example, this behavior was analyzed by looking at the mechanical strength of the interface between two laminated films. Two films were overlapped in a modified lap-shear geometry. The films were pre-treated using a cross-section of available silk film beta sheet treatments and laminated bonds were tested in tension. The results of this experiment are shown in FIG. 12b. As the graph shows, there is a drastic decrease in the bond strength between the films without adhesive when comparing crystallized to uncrystallized pre-treatments. When adhesive is introduced this decrease is less marked, and sufficiently strong bonds are established in all treatment cases.

The results also correlate well with the scanning electron microscope images shown in FIG. 12c. The bottom row shows visible interfacial gaps in the crystallized films on two different length scales, while the top row shows indistinguishable interfaces in almost all cases. Mechanistically, reflow is likely no longer possible in dried crystallized films, unlike the plasticized untreated films, leading to poor intermixing and poor adhesion without the additional adhesive layer.

Notable in the results is an inconsistency in the ultimate strength of the bonds produced by different crystallization techniques. The majority of the samples for each treatment in the glue case broke in the bulk of the material and not at the interface, indicating that the differences in ultimate bond strength are due to the unequal effect of the crystallization techniques on the tensile strength of the material. This correlates well with the hydration state of the crystallized samples, with treatments that produce drier films leading to weaker materials. Regardless, the strength of the bond with the addition of silk adhesive in this case is at least 1100 kPa, which should be more than robust enough for the pocket to survive in an in vivo setting, where shear at the bond interface will likely be minimal. Without wishing to be held to a particular theory, this should allow the manufacture of strong pockets regardless of required pre-fabrication steps. Additionally, control of the crystallinity and localization of the glue layers should allow for spatially controlled adhesive interfaces in multilayer silk constructs.

Behavior of System Components

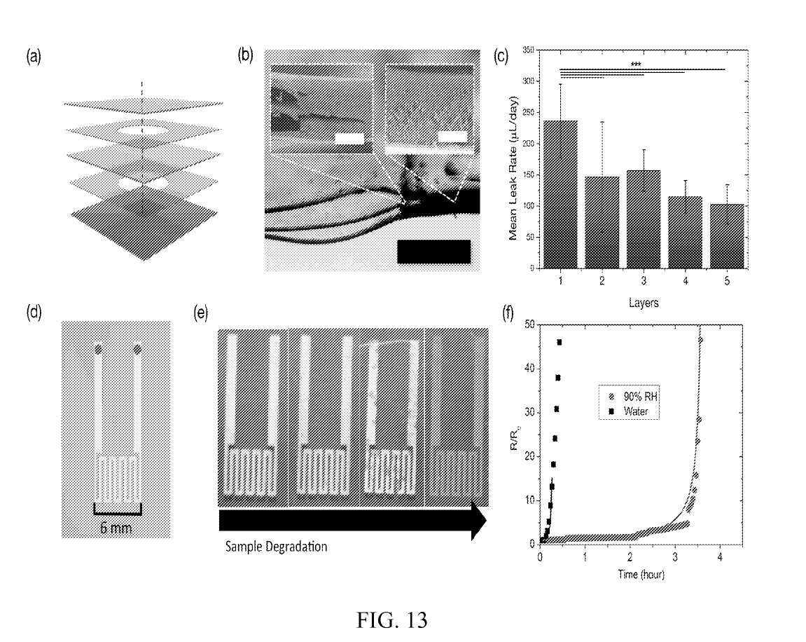

In order to test individual system components, multilayer membranes were fabricated by lamination using the geometry shown in FIG. 13a. The individual crystallized membrane layers were interdigitated with thin uncrystallized adhesive layers that had the center section removed. The stacks were then laminated with pressure applied only to the outer glue containing portion of the films for further spatial control of the interface, leading to uniform membranes with a high degree of interfacial mixing around the edges and negligible adhesion in the center. This structure was confirmed via SEM and optical microscopy, as shown in FIG. 13b.

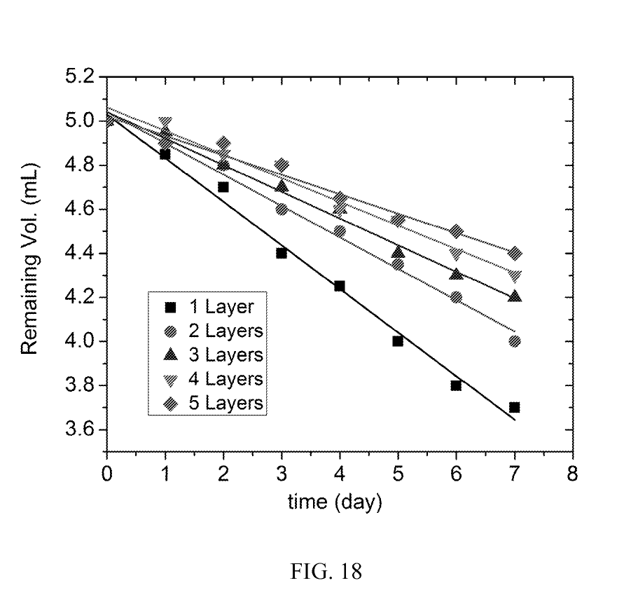

After being mounted to sealed water containing tubes, the samples were exposed to ambient conditions for two weeks, during which water could be lost from the tube by a combination of mass transfer processes. The remaining water volume in the tube followed linear behavior over the two week period as would be expected for mass transfer into an infinite reservoir (FIG. 18). No liquid water was collected outside of the tubes, suggesting evaporative losses only. This indicates that the internal pocket environment contains high humidity air with little to no liquid water, and additionally that water movement through subsequent layers is highly affected by the collapse mechanics of the hydrated films. In cases of direct contact between subsequent layers, water penetration will be much quicker through increased kinetics of hydration of the layer below. Thus, the thickness of the layers will likely play a secondary role to the number of air interfaces in determining device lifetime. The water loss curves were fit linearly to determine the mean leak rate for each sample. These values are compared in FIG. 13c. In all cases, a small absolute magnitude was seen in the evaporative behavior, with a decrease in rate for each subsequent air interface added, showing that multiple air interfaces can serve as an effective control method for degradation rate.

It was expected that the existence of an air interface will also have a direct effect at the device level via the air/device interface. This was investigated through the degradation of magnesium conductors under high relative humidity conditions to mimic the internal pocket environment, in an experiment similar to those conducted in earlier works. Magnesium resistors were fabricated on glass slides according to the dimensions shown in FIG. 13d, and placed either in a high relative humidity (RH) environment or in direct water contact. Images of the degradation behavior of these resistors in high RH are shown in FIG. 13e. Unlike in the water case, here degradation rates were not uniform across the surface, beginning at preferential islands and spreading outward. These islands likely indicate preferential nucleation sites for water adsorption by the magnesium. Eventually full degradation does occur. This behavior is reflected in the kinetics of degradation, shown in FIG. 13f. Degradation follows nearly identical behavior to previous established analytical modeling results, but with a much slower degradation rate. Together, the results of these experiments show that silk (multi)pocket systems slow degradation by a combination of mechanisms that leverage the silk/air/device interface. Slow water penetration due to additional barriers, limited device contact due to pocket architecture, and slower device degradation in high relative humidity environments should all contribute to longer, controllable degradation times of encapsulated magnesium devices.

Proof of Principle and Enhancement of Function

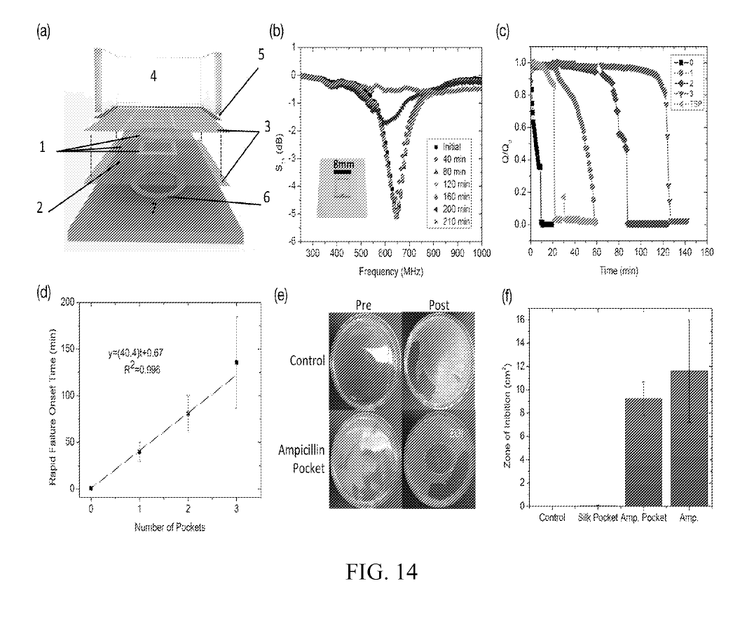

Finally, the entire designed system was combined to test the effectiveness of silk pockets for controlled device degradation. As a proof of principle experiment, simple bilayer metamaterial antennas fabricated in magnesium on silk substrates were degraded and measured in situ. A schematic of the sample design is shown in FIG. 14a. Antenna devices (1, 2) were encapsulated in silk(multi) pockets (3) and affixed to an acrylic well to contain the water exposure to the top-side of the pocket (4, 5), thereby limiting water underneath the device from obscuring the signal. During the experiment, 1 mL of DI water was added to the wells and the resonant response of the encapsulated antenna was monitored at one-minute intervals using the co-localized transceiver antenna (6, 7) until the signal was lost. Characteristic degradation behavior is shown in FIG. 14b. Here we can see that the initial resonance at 650 MHz decreased in amplitude but not in frequency over time, with the exception of a small downfield shift that can be ascribed to swelling of the silk substrate. This is largely a consequence of the metamaterial design, used in this case to simplify analysis.

Antenna quality factors were calculated to monitor the degradation, as is shown in FIG. 14c. As the Figure shows, the degradation exhibited a bimodal behavior in all cases, with an initial phase of little change followed by rapid degradation. This initial phase is likely due to the slow penetration of water into the multi pocket systems, followed by rapid device degradation once wetting occurred. Also tested was a single layer pocket in which the silk protection layers were of equivalent thickness to the total three layer system. This device degraded on a scale comparable to the 1 layer pocket, further supporting the importance multiple air interfaces in slowing device degradation. To further analyze the behavior, the onset time of the rapid phase of degradation was determined for each sample by identifying the point at which degradation exceeded 3% per minute. A comparison of onset times by number of pockets is presented in FIG. 14d. This shows a remarkably linear behavior, wherein each point can be attributed to the addition of an identical silk/air interface into the protection scheme.

Apart from allowing for control and extension of the life of transient devices through its diffusional properties, encapsulation in silk can additionally extend the functionality of the completed device, due to the innate ability of silk to stabilize bioactive compounds. For implantable devices, a common concern is development of infection at the insertion site. If the silk pockets also stabilize antibiotics that will release when the pocket is inserted, the effectiveness and safety of the device can be improved.

To investigate this, silk pockets were fabricated with and without ampicillin doped silk. These pockets were then placed on bacterial lawns and left for 30 minutes. FIG. 14e shows the results of this test. A clear zone of inhibition (ZOI) can be noted around the area where the silk pocket was placed, which is not seen in the control groups. Quantification of the ZOI in FIG. 14f shows equivalent killing by both the pockets and antibiotic solution, and no bacterial death in either control. This indicates that the extension of silk pockets to include bioactive compounds as dopants could allow for subsequent antibiotic delivery along with device insertion, extending the utility of the silk encapsulation layer. This concept could also be further extended to take advantage of the multiple mass transfer rates seen within the multi-pocket system. Here, the device could be used to control the rate of release of multiple bioactive compounds, while simultaneously controlling the rate of water penetration, and thus device degradation.

This Example illustrates certain provided methods of fabricating multilayer structures out of silk fibroin films with controllable interfaces, regardless of the crystallinity and water content of the initial films. Investigation of the silk/air and air/device interfaces allows this method to be adopted for the protection of water-sensitive electronics. In this Example, the silk pocket was introduced as a robust encapsulation strategy for transient magnesium and silicon nanomembrane devices, which will survive in wet environments for controllable periods of time due to the limited penetration of water at the silk/air interface. With the addition of dopant or device triggered transient silk degradation, such devices could see widespread use in the burgeoning field of transient and implantable electronics.

### Equivalents and Scope

Those skilled in the art will recognize, or be able to ascertain using no more than routine experimentation, many equivalents to the specific embodiments of the invention described herein. The scope of the present invention is not intended to be limited to the above Description, but rather is as set forth in the following claims:

1. A multilayered composition comprising
a first silk fibroin layer; and
a second silk fibroin layer,
wherein at least a portion of the first silk fibroin layer is directly adhered to at least a portion of the second silk fibroin layer via a silk-silk interface having a bond strength of at least 500 kPa.

2. The multilayered composition of claim 1, wherein the silk-silk interface has a bond strength of at least 1000 kPa.

3. The multilayered composition of claim 1, wherein the silk-silk interface has a bond strength of at least 1500 kPa.

4. The multilayered composition of claim 1, wherein the silk-silk interface has a bond strength of at least 2000 kPa.

5. The multilayered composition of claim 1, wherein the silk-silk interface has a bond strength of at least 2500 kPa.

6. The multilayered composition of claim 1, wherein the silk-silk interface defines a boundary around non-adhered portions of the first and second silk fibroin layers, thereby defining a pocket.

7. The multilayered composition of any one of claims 1-6, further comprising a third silk fibroin layer wherein at least a portion of the third silk fibroin layer is directly adhered to at least a portion of at least one of the first silk fibroin layer and second silk fibroin layer via a second silk-silk interface.

8. The multilayered composition of claim 7, wherein the second silk-silk interface defines a boundary around the non-adhered portions of at least one of the first and second silk fibroin layers, thereby defining a second pocket.

9. The multilayered composition of any one of claims 1-8, further comprising a third silk fibroin layer and a fourth silk fibroin layer, wherein at least a portion of the third silk fibroin layer is directly adhered to at least a portion of the fourth silk fibroin layer via an additional silk-silk interface to form an additional pocket.

10. The multilayered composition of claim 9, wherein the first silk fibroin layer and second silk fibroin layer are encapsulated within the additional pocket.

11. The multilayered composition of any one of claims 6-10, wherein the pocket contains or is filled with a gas.

12. The multilayered composition of claim 11, wherein the gas is selected from air, nitrogen, argon, and CO₂.

13. The multilayered composition of any one of claims 6-10, wherein the pocket contains or is filled with a liquid.

14. The multilayered composition of claim 13, wherein the liquid is selected from an aqueous liquid and an organic liquid.

15. The multilayered composition of any one of claims 6-10, wherein the pocket contains or is filled by an active agent.

16. The multilayered composition of claim 15, wherein the active agent is a biologically active agent.

17. The multilayered composition of claim 15, wherein the active agent is an electrically active agent.

18. The multilayered composition of any one of the above claims, wherein the multilayered composition comprises a plurality of pockets.

19. A method for bonding a first silk fibroin layer with a second silk fibroin layer via a silk-silk interface, the method comprising the steps of:
contacting a first silk fibroin layer with a second silk fibroin layer; and
inducing reflow of silk fibroin of the first silk fibroin layer and silk fibroin of the second silk fibroin layer to generate a silk-silk interface with a bond strength of at least 500 kPa.

20. The method of claim 19, wherein the step of inducing comprises treating with heat, pressure, or combination thereof for a duration of time sufficient to induce reflow of silk fibroin at the silk-silk interface.

21. The method of claim 20, wherein the heat is between 75-150° C.

22. The method of claim 20, wherein the duration of time is between 1-120 seconds.

23. The method of claim 22, wherein the duration of time is between 5-30 seconds.

24. The method of any one of claims 19-23, wherein the first silk fibroin layer is not annealed.

25. The method of any one of claims 19-24, wherein the second silk fibroin layer is not annealed.

26. The method of any one of claims 19-25, wherein the first silk fibroin layer has a first initial crystallinity and first initial water content, and the second silk fibroin layer has a second initial crystallinity and second initial water content;
wherein the first initial crystallinity and the second initial crystallinity are different; and
wherein the first initial water content and the second initial water content are different.

27. A multilayered composition comprising
a first silk fibroin layer;
a second silk fibroin layer; and
a device;
wherein at least a portion of the first silk fibroin layer is directly adhered to at least a portion of the second silk fibroin layer via a silk-silk interface, and wherein the device is located, at least in part, between the first silk fibroin layer and the second silk fibroin layer.

28. The multilayered composition of claim 27, wherein the silk-silk interface has a bond strength of at least 500 kPa.

29. The multilayered composition of claim 27, wherein the silk-silk interface has a bond strength of at least 1000 kPa.

30. The multilayered composition of claim 27, wherein the silk-silk interface has a bond strength of at least 1500 kPa.

31. The multilayered composition of claim 27, wherein the silk-silk interface has a bond strength of at least 2000 kPa.

32. The multilayered composition of claim 27, wherein the silk-silk interface has a bond strength of at least 2500 kPa.

33. The multilayered composition of claim 27, wherein the silk-silk interface defines a boundary around non-adhered portions of the first and second silk fibroin layers, thereby defining a pocket.

34. The multilayered composition of any one of claims 27-33, further comprising a third silk fibroin layer wherein at least a portion of the third silk fibroin layer is directly adhered to at least a portion of at least one of the first silk fibroin layer and second silk fibroin layer via a second silk-silk interface.

35. The multilayered composition of claim 34, wherein the second silk-silk interface defines a boundary around the non-adhered portions of at least one of the first and second silk fibroin layers, thereby defining a second pocket.

36. The multilayered composition of any one of claims 27-35, further comprising a third silk fibroin layer and a fourth silk fibroin layer, wherein at least a portion of the third silk fibroin layer is directly adhered to at least a portion of the fourth silk fibroin layer to form an additional silk-silk interface and an additional pocket.

37. The multilayered composition of claim 36, wherein the first silk fibroin layer and second silk fibroin layer are encapsulated within the additional pocket.

38. The multilayered composition of any one of claims 33-37, wherein the pocket contains or is filled with a gas.

39. The multilayered composition of claim 38, wherein the gas is selected from air, nitrogen, argon, and CO₂.

40. The multilayered composition of any one of claims 33-37, wherein the pocket contains or is filled with a liquid.

41. The multilayered composition of claim 40, wherein the liquid is selected from an aqueous liquid and an organic liquid.

42. The multilayered composition of any one of claims 33-37, wherein the pocket contains or is filled by an active agent.

43. The multilayered composition of claim 42, wherein the active agent is a biologically active agent.

44. The multilayered composition of claim 42, wherein the active agent is an electrically active agent.

45. The multilayered composition of any one of claims 33-44, wherein the multilayered composition comprises a plurality of pockets.

46. The multilayered composition of any one of claims 27-45, further comprising a bioactive compound.

47. The multilayered composition of claim 46, wherein the bioactive compound is located substantially within the pocket.

48. The multilayered composition of claim 46 or 47, wherein the bioactive compound is selected from: an antibiotic, an antiviral, an antifungal, an anti-thrombotic, a fragrance, a vitamin, a nutrient, a food, a retroviral agent, a nanoparticle, a quantum dot, and a growth factor.

49. The multilayered composition of any one of claims 27-48, wherein the device is selected from: a sensor, a transmitter, an antenna, a transistor, an LED, and a receiver.

50.-70. (canceled)

71. A method comprising
providing a multilayered composition according to any one of claims 27-49;
placing the multilayered composition in an environment; and
activating the device.

72. The method of claim 71, wherein the device degrades over a period of time.

73. The method of claim 72, wherein the period of time is at least one day.

74. The method of claim 72, wherein the period of time is at least one week.

75. The method of claim 72, wherein the period of time is at least one month.
